(19) United States
(12) Patent Application Publication
(10) Pub. No.: US 2003/0096415 A1
(43) Pub. Date: May 22, 2003

(54) Infection with chimaeric adenoviruses of cells negative for the adenovirus serotype 5 Coxsacki adenovirus receptor (CAR)

(76) Inventors: Menzo Havenga, Alphen aan den Rijin (NL); Ronald Vogels, Linchoten (NL)

Correspondence Address: TRASK BRITT, P.O. BOX 2550, SALT LAKE CITY, UT, 84110, US

(21) Appl. No.: 10/040,949

(22) Filed: Jan. 7, 2002

Related U.S. Application Data
(63) Continuation of application No. PCT/NL00/00481, filed on Jul. 7, 2000.

(51) Int. Cl. C12N 15/861; A61K 48/00; C12N 7/00

(52) U.S. Cl. 435/456; 435/235.1; 435/320.1; 424/93.2

(57) ABSTRACT

A method for delivering a nucleic acid of interest to a host cell (“gene therapy”) using a gene delivery vehicle based on adenoviral material. The gene delivery vehicle delivers the nucleic of acid of interest to the host cell by associating with a binding site and/or a receptor present on adenovirus serotype 5 Coxsacki adenovirus receptor (“CAR”)-negative cells. The binding site and/or receptor is a binding site for adenovirus subgroup D and/or adenovirus subgroup F. Associated methods and pharmaceutical compositions are also disclosed.

### CROSS-REFERENCE TO RELATED APPLICATION

This application is a continuation of International Application Number PCT/NL00/00481 filed on Jul. 7, 2000 designating the United States of America, International Publication No. WO 01/04334 (Jan. 18, 2001), the contents of the entirety of which is incorporated by this reference.

### TECHNICAL FIELD

The invention relates to the field of molecular genetics and medicine. In particular, the invention relates to the field of gene therapy, especially gene therapy involving adenovirus.

### BACKGROUND

In gene therapy, genetic information is delivered to a host cell in order to either correct (supplement) a genetic deficiency in said cell, or to inhibit an unwanted function in said cell, or to eliminate said host cell. Of course the genetic information can also be intended to provide the host cell with a wanted function, for instance to supply a secreted protein to treat other cells of the host, etc.

Thus, basically three different approaches to gene therapy exist. The first is directed towards compensating a deficiency present in a (mammalian) host. The second is directed towards the removal or elimination of unwanted substances (organisms or cells). The third is directed towards providing a cell with a desired function.

For the purposes of gene therapy, adenoviruses have been proposed as a suitable vehicle to deliver genes to the host. Gene-transfer vectors derived from adenoviruses (so-called “adenoviral vectors”) have a number of features that make them particularly useful for gene transfer. 1) Adenovirus biology is characterized in detail. 2) Adenovirus is generally not associated with severe human pathology. 3) Adenovirus is extremely efficient in introducing its DNA into the host cell. 4) Adenovirus can infect a wide variety of cells and has a broad host-range. 5) Adenovirus can be produced at high virus titers in large quantities. 6) Adenovirus can be rendered replication defective by deletion of the early-region 1 (E1) of the viral genome (Brody et al., 1994).

However, there are still drawbacks associated with the use of adenoviral vectors especially the well investigated serotypes of subgroup C adenoviruses. These serotypes require the presence of the Coxsacki adenovirus receptor (CAR) on cells for successful infection. Although this protein is expressed by many cells and established cell lines, this protein is absent for many other primary cells and cell lines making the latter cells difficult to infect with serotypes 1, 2, 5, and 6.

The adenovirus genome is a linear double-stranded DNA molecule of approximately 36000 base pairs. The adenovirus DNA contains identical Inverted Terminal Repeats (ITR) of approximately 90-140 base pairs with the exact length depending on the serotype. The viral origins of replication are within the ITRs exactly at the genome ends.

Most adenoviral vectors currently used in gene therapy have a deletion in the E1 region, where novel genetic information can be introduced. The E1 deletion renders the recombinant virus replication defective (Levrero et al, 1991). It has been demonstrated extensively that recombinant adenovirus, in particular serotype 5 is suitable for efficient transfer of genes in vivo to the liver, the airway epithelium and solid tumours in animal models and human xenografts in immunodeficient mice (Bout, 1996; Blaese et al., 1995). Thus, preferred methods for in vivo gene transfer into target cells make use of adenoviral vectors as gene delivery vehicles.

At present, six different subgroups of human adenoviruses have been proposed which in total encompasses 51 distinct adenovirus serotypes. Besides these human adenoviruses an extensive number of animal adenoviruses have been identified (see Ishibashi et al, 1983).

A serotype is defined on the basis of its immunological distinctiveness as determined by quantitative neutralisation with animal antisera (horse, rabbit). If neutralisation shows a certain degree of cross-reaction between two viruses, distinctiveness of serotype is assumed if A) the hemagglutinins are unrelated, as shown by lack of cross-reaction on hemagglutination-inhibition, or B) substantial biophysical/biochemical differences in DNA exist (Francki et al, 1991). The nine serotypes identified last (42-51) were isolated for the first time from HIV-infected patients (Hierholzer et al 1988; Schnurr et al 1993; De Jong et al 1998). For reasons not well understood, most of such immuno-compromised patients shed adenoviruses that were rarely or never isolated from immuno-competent individuals (Hierholzer et al 1988, 1992; Khoo et al, 1995, De Jong et al, 1998).

The adenovirus serotype 5 is most widely used for gene therapy purposes. Similar to serotypes 2, 4 and 7, serotype 5 has a natural affiliation towards lung epithelia and other respiratory tissues. In contrast, it is known that, for instance, serotypes 40 and 41 have a natural affiliation towards the gastrointestinal tract. For a detailed overview of the disease association of the different adenovirus serotypes see table 1. The underlying reason for the different natural affiliations of serotypes towards specific organs can be manifold. Such reasons may include but need not be limited to the observation that serotypes differ in the route of infection or make use of different receptor molecules or internalisation pathways or that a serotype can infect many tissues/organs but it can only replicate in one organ because of the requirement of certain cellular factors for replication. As mentioned before, it is presently unknown which mechanisms are responsible for the observed differences in human disease association.

One of the problems associated with the development of effective Gene Therapy protocols for the treatment of disease is the limitation of the current vectors to effectively transduce cells in vivo. One of the most effective ways to deliver foreign genetic material to cells in vivo is through the use of adenovirus vectors. Although, the vector system is very efficient the current adenovirus vector technology has its limitation. Specifically were certain cell types need to be transduced that are normally not very efficiently transduced by Adenovirus 2 or 5. Examples of such relatively resistant cell types include endothelial cells, smooth muscle cells, dendritic cells, neuronal cells, glial cells, synovical cells, primary fibroblasts, cells from the amniotic fluid, hemopoietic stem cells, and monocytic/macrophage cells etc. Thus in one aspect the invention provides a method for delivering a nucleic acid of interest to a host cell by means of a gene delivery vehicle based on adenoviral material, whereby said gene delivery vehicle delivers the nucleic acid to the host cell by associating with a binding site and/or a receptor present on CAR-negative cells, said binding site and/or receptor being a binding site and/or a receptor for adenovirus subgroups D and/or F. The method may advantageously be used to efficiently transduce cells both in vitro and in vivo.

The present invention was made during research with chimaeric adenoviruses. Said chimaeric adenoviruses comprising capsids derived from adenovirus 5 of which at least part of the adenovirus 5 fiber protein was replaced by a fiber protein from a different adenovirus serotype. It was observed that chimaeric adenoviruses comprising fiber protein from adenovirus serotypes belonging to subgroup D or subgroup F were capable of efficiently transducing CAR negative target cells.

Adenovirus 2 and 5 belong to adenovirus subgroup C. Together with the adenoviruses of subgroups A, D-F, the subgroup C adenoviruses were before the present invention thought to attach to cells via the Coxsacki adenovirus receptor (CAR) (Roelvink et al, 1998).

It has been shown that adenoviruses of subgroup B such as Ad3 bind to a different receptor than CAR (Defer et al, 1990). Likewise, it was demonstrated that receptor specificity could be altered by exchanging the Ad3 with the Ad 5 knob protein, and vice versa (Krasnykh et al, 1996; Stevenson et al, 1995, 1997).

A host cell may be any host cell as long at it comprises a binding site and/or a receptor present on CAR-negative cells, said binding site and/or receptor being a binding site and/or a receptor for adenovirus subgroups D and/or F. Preferably, said cell is a human cell. Said cell may be a cell present in a culture dish or be part of a whole organism.

Preferably said CAR-negative cells are hemopoietic cells or amniotic fluid cells or derivatives thereof. Preferably, said CAR-negative hemopoietic cells are K562 cells. Preferably, said CAR-negative amniotic fluid cells are amniotic villi or chorion villi cells or derivatives thereof.

A gene delivery vehicle according to the invention may be any vehicle capable of transferring nucleic acid into cells. Preferably, said gene delivery vehicle is a viral vector particle, more preferably said gene delivery vehicle is an adenoviral vector particle. The word gene in the term gene delivery vehicle does not reflect a situation wherein always an entire gene is delivered by said vehicle. The word gene in this respect merely reflects the presence of a nucleic acid of interest. Said nucleic acid may comprise an entire gene, an artificial sequence, a recombinant nucleic acid, a protein coding domain, a cDNA, a sequence coding for anti-sense RNA, mRNA and/or other kind of nucleic acid.

Suitable adenovirus material may comprise an adenovirus capsid or a functional part, derivative and/or analogue thereof. Said adenovirus capsid preferably comprises an adenovirus subgroup D or subgroup F capsid, or a functional part, derivative and/or analogue thereof. Said adenovirus capsid may also be a chimaeric capsid comprising proteins or parts thereof from at least two different adenovirus serotypes or derivatives and/or analogues thereof. Preferably, at least part of a fiber protein of said chimaeric capsid is derived from an adenovirus of subgroup D and/or subgroup F or a functional derivative and/or analogue thereof. Preferably, capsid proteins other then said part of a fiber protein, are derived from an adenovirus of subgroup C, preferably of adenovirus 5 or adenovirus 2. Suitable derivatives of said adenovirus capsids may, among other, be obtained through so-called silent amino-acid substitution in one or more capsid proteins.

Preferably, said adenovirus material comprises at least part of an adenovirus fiber protein. Preferably, said adenovirus fiber protein is derived from an adenovirus of subgroup D or subgroup F or a functional part, derivative and/or analogue thereof. Preferably, said part of a fiber protein is a part involved in binding to a receptor and/or a binding site on a target cell. Typically, but not necessarily said part of an adenovirus fiber protein involved in binding to a receptor and/or a binding site on a target cell is a part of the knob. Adenovirus fiber protein comprises at least three functional regions. One region, the base, is responsible for anchoring the fiber to a penton base of the adenovirus capsid. Another region, the knob, is typically associated with receptor recognition whereas the shaft region functions as a spacer separating the base from the knob. Various regions may also have other functions. For instance, the shaft is presumably also involved in target cell specificity. Each of the regions mentioned above may be used to define a part of a fiber. However, regions of a fiber may also be identified in another way. For instance the knob region comprises of a receptor binding region and a shaft binding region. The base region comprises of a penton base binding region and a shaft binding region. Moreover, the shaft comprises of repeated stretches of amino acids. Each of these repeated stretches may be a part. A receptor and/or binding site binding part of a fiber protein may be a single region of a fiber protein or a functional part thereof, or a combination of regions or parts thereof of at least one fiber protein, wherein said receptor and/or binding site binding part of a fiber protein, either alone or in combination with one or more other proteins of a adenovirus capsid, determines the efficiency with which a gene delivery vehicle can transduce a given cell or cell type, preferably but not necessarily in a positive way. Needless to say that said fiber and/or a capsid may comprise further modifications to adapt the fiber protein and/or the capsid to specific other needs, which a person skilled in the art will be capable of doing.

A receptor and/or a binding site for adenovirus subgroups D and/or F may be any kind of molecule capable of associating with an adenovirus of subgroup D and/or F. In and/or on the surface of a cell, said receptor and/or binding site must be able to associate with said adenovirus of subgroup D and/or F provided to said cell. Said receptor and/or binding site may be part of a complex present in and/or on said cell. Said receptor and/or binding site does not need to be able to associate with an adenovirus of subgroup D and/or F all the time as long as it is capable of doing so some of the time. Said receptor and/or binding site may further also be a receptor and/or binding site for another virus and/or gene delivery vehicle, although this does not have to be so. A person skilled in the art may want to determine whether an adenovirus serotype belonging to another subgroup than D and/or F can also utilise the receptor and/or binding site for adenovirus subgroups D and/or F.

In another aspect the invention provides the use of a gene delivery vehicle comprising a nucleic acid of interest and comprising adenoviral material involved in binding to a host cell, said material being from a subgroup D and/or F adenovirus, in delivering said nucleic acid of interest to a CAR-negative cell. With the knowledge of a novel pathway for the transduction of cells using adenovirus material it becomes possible to approach this novel pathway also through other means then said material derived from a subgroup D and/or F. A person skilled in the art recognises this and will be able to devise means to accomplish this for instance through the use of antibodies directed toward a crucial component of said pathway, together with a membrane fusion peptide. Such means and methods are also within the scope of the invention.

In another aspect the invention provides a gene delivery vehicle being a chimaera based on at least two adenoviruses, whereby a cell recognising element of said gene delivery vehicle is based on adenoviral material from a subgroup D and/or F adenovirus, which material confers the capability of infecting CAR negative cells.

Preferably, said adenoviral material is based on the fiber, penton and/or hexon proteins of a subgroup D and/or subgroup F adenovirus.

To date, six different subgroups of human adenoviruses have been proposed which in total encompasses 51 distinct adenovirus serotypes. A serotype is defined on the basis of its immunological distinctiveness as determined by quantitative neutralisation with animal antisera (horse, rabbit). If neutralisation shows a certain degree of cross-reaction between two viruses, distinctiveness of serotype is assumed if A) the hemagglutinins are unrelated, as shown by lack of cross-reaction on hemagglutination-inhibition, or B) substantial biophysical/biochemical differences in DNA exist (Francki et al, 1991). The nine serotypes identified last (42-51) were isolated for the first time from HIV-infected patients (Hierholzer et al 1988; Schnurr et al 1993;). For reasons not well understood, most of such immune-compromised patients shed adenoviruses that were rarely or never isolated from immune-competent individuals (Hierholzer et al 1988, 1992; Khoo et al, 1995, De Jong et al, 1998). The usefulness of these adenoviruses or cross-immunising adenoviruses to prepare gene delivery vehicles may be seriously hampered, since the individual to which the gene delivery vehicle is provided, will raise a neutralising response to such a vehicle before long.

There is thus a need in the field of gene therapy to provide gene delivery vehicles, preferably based on adenoviruses, which do not encounter pre-existing immunity and/or which are capable of avoiding or diminishing neutralising antibody responses. Thus preferably, a gene delivery vehicle of the invention further comprises an element from adenovirus 35 or a functional equivalent thereof, responsible for at least partially avoiding an immune response against adenovirus 35. A functional equivalent/homologue of adenovirus 35 (element) for the purpose of the present invention is an adenovirus (element) which, like adenovirus 35, encounters pre-existing immunity in less than about 10% of the hosts, at least in a significant geographic region of the world, to which it is administered for the first time, or which is capable in more than about 90% of the hosts, at least in a significant geographic region of the world, to which it is administered to avoid or diminish the immune response. Typical examples of such adenoviruses are adenovirus serotypes 34, 26 and 48.

In another embodiment a gene delivery vehicle according to the invention comprises an element of adenovirus 16 or a functional equivalent thereof, which element confers said virus with an enhanced capability to infect smooth muscle cells and/or synoviocytes. A functional equivalent of an element of adenovirus 16 in this respect is an element from another subgroup B virus. Preferably, said element is a tissue tropism determining part of a fiber protein. Typically, a tissue tropism determining part of an adenovirus fiber protein is a part that influences the transduction efficiency of a cell.

For Gene Therapeutic purposes one typically does not want an adenovirus batch to be administered to a host cell which contains replication competent adenovirus, although this is not always true. In general therefor it is desired to omit a number of genes (but at least one) from the adenoviral genome on the vector encoding the virus and to supply these genes in the genome of the cell in which the vector is brought to produce adenovirus. Such a cell is usually called a packaging cell. The invention thus also provides a packaging cell for producing an adenovirus according to the invention, comprising in trans, all elements necessary for adenovirus production not present on the adenoviral vector according to the invention. Typically vector and packaging cell have to be adapted to one another in that they have all the necessary elements, but that they do not have overlapping elements which lead to replication competent virus by recombination. In a preferred embodiment said packaging cell is, or is derived from PER.C6 (ECCAC deposit number 96022940).

In another embodiment, a gene delivery vehicle according to the invention comprises an adenovirus vector. Said adenovirus vector may be a classical adenovirus vector, a minimal adenovirus vector or an integrating adenovirus such as an Ad/AAV chimaeric vector, a retro-adenovirus or a transposon-adenovirus or yet another different kind of adenovirus vector. With an integrating adenovirus vector for the purpose of the invention is meant a vector comprising nucleic acid derived from an adenovirus and further comprising means for the integration of at least part of the nucleic acid of said vector into the host cell genome. Said means are preferably derived from a nucleic acid with the inherent capacity to integrate into the host cell genome. Such nucleic acid with the inherent capacity to integrate into the host cell genome may derived from a transposon or transposon-like element, a retrovirus and/or an adeno-associated virus or a different virus with the capacity to integrate nucleic acid into the host cell genome.

In a preferred embodiment said adenovirus vector comprises nucleic acid encoding at least a receptor and/or binding site determining part of a fiber protein of an adenovirus of subgroup D or subgroup F. In a preferred embodiment the invention provides a method for producing said adenovirus vector, comprising welding together, preferably through homologous recombination, two nucleic acid molecules comprising partially overlapping sequences wherein said overlapping sequences allow essentially only one homologous recombination which leads to the generation of a physically linked nucleic acid comprising at least two functional adenovirus inverted terminal repeats, a functional encapsulation signal and a nucleic acid of interest or functional parts, derivatives and/or analogues thereof. In a preferred embodiment at least one of said at least two nucleic acid molecules comprises nucleic acid encoding at least a receptor and/or binding site determining part of a fiber protein of an adenovirus of subgroup D or subgroup F. An important aspect in this embodiment of the invention is that said partially overlapping sequences allow essentially only homologous recombination leading to the generation of a functional adenovirus vector capable of being replicated and packaged into adenovirus particles in the presence of the required transacting functions. With essentially only one is meant that said overlapping sequences in each nucleic acid comprise essentially only one continuous sequence wherein homologous recombination leading to the generation of a functional adenovirus may occur. Within said continuous sequence the actual number of homologous recombination events may be higher than one. Non continuous overlapping sequences are not desired because they reduce the reliability of said method. Non continuous overlapping sequences are also not desired because they reduce the overall efficiency of said method, presumably due to the generation of undesired homologous recombination products.

A preferred embodiment of the invention provides a method for generating an adenovirus vector wherein both of said nucleic acid molecules comprise only one adenovirus inverted terminal repeat or a functional part, derivative and/or analogue thereof. In one aspect one or both of said two nucleic acid molecules have undergone modifications prior to said welding together. Said modification may include the welding together of different nucleic acid molecules leading to the generation of one or both of said two nucleic acid molecules. In a preferred embodiment said different nucleic acids are welded together through homologous recombination of partially overlapping sequences. In a further aspect said welding together is performed in a cell or a functional part, derivative and/or analogue thereof. Preferably said cell is a mammalian cell. More preferably, said welding together is performed in a cell expressing E1-region encoded proteins. Preferably said cell is a PER.C6 cell (ECACC deposit number 96022940) or a derivative thereof. In a preferred embodiment said nucleic acid molecules are not capable of replicating in said mammalian cell prior to said welding together. Said replication is undesired since it reduces the reliability of the methods of the invention presumably through providing additional targets for undesired homologous recombination. Said replication is also not desired because it reduces the efficiency of the methods of the invention presumably because said replication competes for substrate or adenovirus transacting functions with the replication of said adenovirus vector.

In a preferred embodiment, one of said nucleic acid molecules is relatively small and the other is relatively large. This configuration is advantageous because it allows easy manipulation of said relatively small nucleic acid molecule allowing for example the generation of a large number of small nucleic acid molecules comprising different nucleic acid of interest for instance for the generation of an adenovirus vector library. Said configuration is also desired because it allows the production of a large batch of quality tested large nucleic acid molecule. The amplification of large nucleic acid molecules for instance in bacteria is difficult in terms of obtaining sufficient amounts of said large nucleic acid. The amplification of large nucleic acid molecules for instance in bacteria is also difficult to control because a small modification of said large nucleic acid is not easily detected. Moreover, for reasons not quite understood some large vectors are more stable in bacteria or yeasts than others. Said configuration however, allows the generation of a standard batch of a large nucleic acid molecule which can be thoroughly tested, for instance through generating a control adenovirus of which the efficiency and the reliability of production is known, and determining said parameters of a new batch of large nucleic acid molecule. Once validated said batch may be used for the generation of a large number of different adenovirus vectors through combining said large molecule with a large number of different small nucleic acid molecules. Said system therefore also allows for the selection and/or manipulation of vectors comprising a large nucleic acid molecule of the invention to allow a suitable yield of intact large nucleic acid.

In another embodiment said cell comprising nucleic acid encoding E1-region proteins further comprises a nucleic acid encoding an adenovirus E2-region and/or an adenovirus E4-region protein. Preferably, said cell further comprising nucleic acid encoding an adenovirus E2-region and/or an adenovirus E4-region protein is a derivative of PER.C6.

In another aspect the invention provides a receptor and/or a binding site for adenoviruses type D and/or F, present on or associated with CAR negative cells. Preferably said receptor and/or a binding site is present on K562 cells, amniotic fluid derived cells and/or primary fibroblast cells.

In yet another aspect, the invention provides the use of receptor and/or a binding site for adenoviruses type D and/or F, present in and/or on a cell, for the delivery nucleic acid to said cell.

In yet another embodiment the invention provides the use of a gene delivery vehicle according to anyone of claims 1-14, in a pharmaceutical.

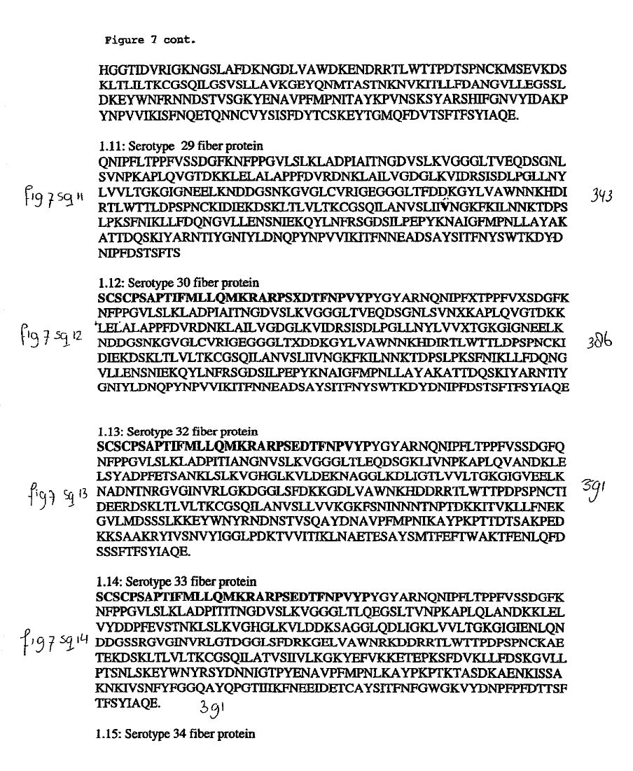

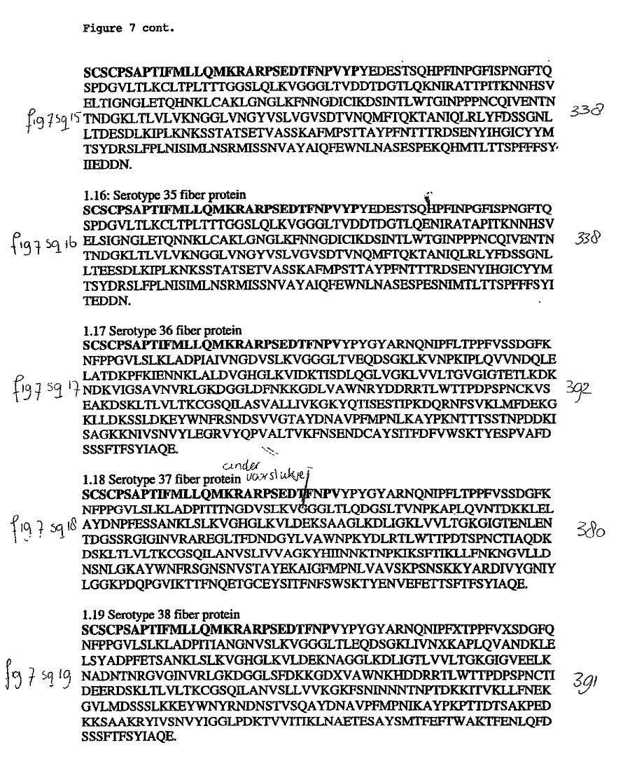

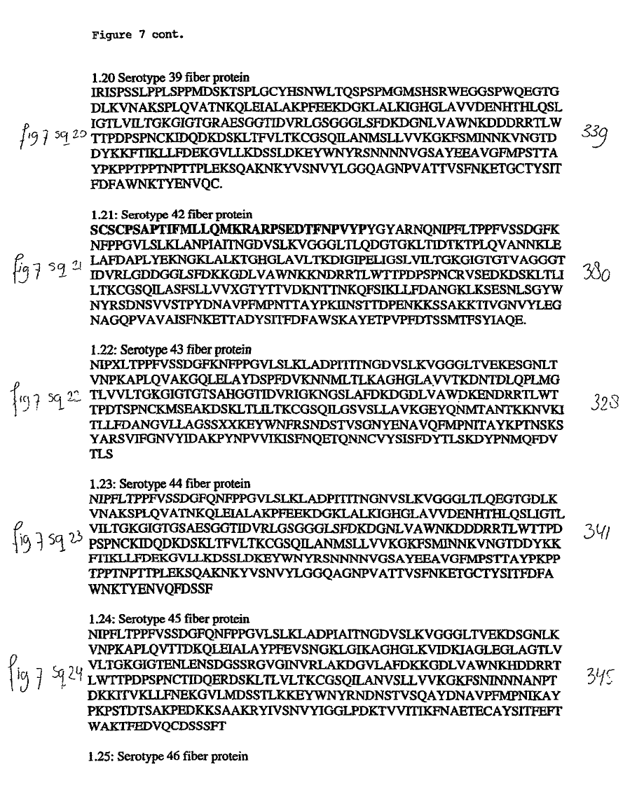

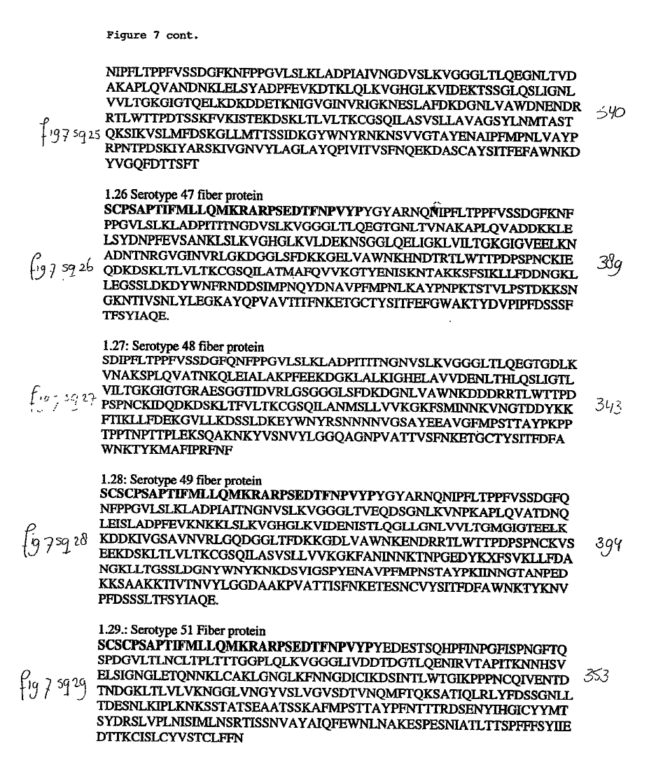

In another aspect the invention provides a capsid protein derived from a subgroup D and/or a subgroup F adenovirus or a functional part, derivative and/or analogue thereof. Preferably, said protein is a fiber protein. The invention further provides a nucleic acid encoding a capsid protein of the invention. Preferably, said nucleic acid comprises a fiber sequence from a subgroup D and/or a subgroup F as depicted in FIG. 7.

### DETAILED DESCRIPTION

It has been demonstrated in mice that upon in vivo systemic delivery of recombinant adenovirus serotype 5 for gene therapy purposes approximately 99% of the virus is trapped in the liver (Herz et al, 1993). Therefore, alteration of the adenovirus serotype 5 host cell range to be able to target other organs in vivo is a major interest of the invention.

The initial step for successful infection is binding of adenovirus to its target cell, a process generally thought to be mediated through fiber protein. The fiber protein has a trimeric structure (Stouten et al, 1992) with different lengths depending on the virus serotype (Signas et al 1985; Kidd et all 1993). Different serotypes have polypeptides with structurally similar N and C termini, but different middle stem regions. N-terminally, the first 30 amino acids are involved in anchoring of the fiber to the penton base (Chroboczek et al, 1995), especially the conserved FNPVYP region in the tail (Arnberg et al 1997). The C-terminus, or knob, is generally thought to be responsible for initial interaction with the cellular adenovirus receptor. After this initial binding secondary binding between the capsid penton base and cell-surface integrins is proposed to lead to internalisation of viral particles in coated pits and endocytosis (Morgan et al, 1969; Svensson et al, 1984; Varga et al, 1992; Greber et al, 1993; Wickham et al, 1994).

Integrins are αβ-heterodimers of which at least 14 α-subunits and 8 β-subunits have been identified (Hynes et al, 1992). The array of integrins expressed in cells is complex and will vary between cell types and cellular environment. Although the knob contains some conserved regions, between serotypes, knob proteins show a high degree of variability, indicating that different adenovirus receptors might exist. For instance, it has been demonstrated that adenoviruses of subgroup C (Ad2, Ad5) and adenoviruses of subgroup B (Ad3) bind to different receptors (Defner et al, 1990). By using baculovirus produced soluble CAR as well as adenovirus serotype 5 knob protein, Roelvink et all concluded via interference studies that all adenovirus serotypes, except serotypes of subgroup B, enter cells via CAR (Roelvink et al, 1998). The latter, which is now generally accepted in the field, if valid should thus limit the complexity of using different serotypes for gene therapy purposes.

Besides the involvement in cell binding, the fiber protein also contains the type specific γ-antigen, which together with the ε-antigen of the hexon determines the serotype specificity. The γ-antigen is localised on the fiber and it is known that it consists of 17 amino acids (Eiz et al, 1997). The anti-fiber antibodies of the host are therefore directed to the trimeric structure of the knob. To obtain re-directed infection of recombinant adenovirus serotype 5, several approaches have been or still are under investigation. Wickham et al has altered the RGD (Arg, Gly, Asp) motif in the penton base which is believed to be responsible for the αᵥβ₃ and αᵥβ₅ integrin binding to the penton base. They have replaced this RGD motif by another peptide motif which is specific for the α₄β₁ receptor. In this way targeting the adenovirus to a specific target cell could be accomplished (Wickham et al, 1995, 1996). Krasnykh et al has made use of the HI loop available in the knob. This loop is, based on x-ray crystallographics, located on the outside of the knob trimeric structure and therefore is thought not to contribute to the intramolecular interactions in the knob (Krasnykh et al, 1998). However, complete CAR independent infection was not observed.

It is an object of the present invention to provide a method and means by which an adenovirus can infect cells negative for the CAR protein. Therefore, the generation of chimaeric adenoviruses based on adenovirus serotype 5 with a modified fiber gene is described. For this purpose, two or three plasmids, which together contain the complete adenovirus serotype 5 genome, were constructed. From a plasmid the DNA encoding the adenovirus serotype 5 fiber protein was essentially removed and replaced by linker DNA sequences which facilitate easy cloning. This plasmid subsequently served as template for the insertion of DNA encoding for fiber protein derived from different adenovirus serotypes (human or animal). The DNAs derived from the different serotypes were obtained using the polymerase chain reaction technique in combination with (degenerate) oligo-nucleotides. At the former E1 location in the genome of adenovirus serotype 5, any gene of interest can be cloned. A single transfection procedure of the two or three plasmids together resulted in the formation of a recombinant chimaeric adenovirus. Although successful introduction of changes in the adenovirus serotype 5 fiber and penton-base have been reported, the complex structure of knob and the limited knowledge of the precise amino acids interacting with CAR render such targeting approaches laborious and difficult. To overcome the limitations described above we used pre-existing adenovirus fibers to maximise the chance of obtaining recombinant adenovirus which can normally assemble in the nucleus of a producer cell and which can be produced on pre-existing packaging cells. By generating a chimaeric adenovirus serotype 5 based fiber library containing fiber proteins of all other human adenovirus serotypes, we have developed a technology which enables rapid screening for a recombinant adenoviral vector with preferred infection characteristics.

In one aspect this invention describes chimaeric adenoviruses and methods to generate these viruses that have an tropism different from that of adenovirus serotype 5. This chimaeric adenovirus serotype 5 is able to infect cell types which do not express the CAR protein much more efficiently both in vitro and in vivo than the adenovirus serotype 5. Such cells include but are not limited to endothelial cells, smooth muscle cells, dendritic cells, neuronal cells, glial cells, synovical cells, primary fibroblasts, cells from the amniotic fluid, hemopoietic stem cells, and monocytic/macrophage cells etc.

In another aspect the invention describes the construction and use of plasmids consisting of distinct parts of adenovirus serotype 5 in which the gene encoding for fiber protein has been replaced with DNA derived from alternative human or animal serotypes. This set of constructs, in total encompassing the complete adenovirus genome, allows for the construction of unique chimaeric adenoviruses customised for transduction of particular cell types or organ(s).

In all aspects of the invention the chimaeric adenoviruses may, or may not, contain deletions in the E1 region and insertions of heterologous genes linked either or not to a promoter. Furthermore, chimaeric adenoviruses may, or may not, contain deletions in the E3 region and insertions of heterologous genes linked to a promoter. Furthermore, chimaeric adenoviruses may, or may not, contain deletions in the E2 and/ or E4 region and insertions of heterologous genes linked to a promoter. In the latter case E2 and/or E4 complementing cell lines are required to generated recombinant adenoviruses.

### EXAMPLE 1

### Generation of Adenovirus Serotype 5 Genomic Plasmid Clones

The complete genome of adenovirus serotype 5 has been cloned into various plasmids or cosmids to allow easy modification of parts of the adenovirus serotype 5 genome, while still retaining the capability to produce recombinant virus. For this purpose the following plasmids were generated:

1. pBr/Ad.Bam-rITR (ECACC Deposit P97082122)

In order to facilitate blunt end cloning of the ITR sequences, wild-type human adenovirus type 5 (Ad5) DNA was treated with Klenow enzyme in the presence of excess dNTPs. After inactivation of the Klenow enzyme and purification by phenol/chloroform extraction followed by ethanol precipitation, the DNA was digested with BamHI. This DNA preparation was used without further purification in a ligation reaction with pBr322 derived vector DNA prepared as follows: pBr322 DNA was digested with EcoRV and BamHI, dephosphorylated by treatment with TSAP enzyme (Life Technologies) and purified on LMP agarose gel (SeaPlaque GTG). After transformation into competent E. coli DH5a (Life Techn.) and analysis of ampicilline resistant colonies, one clone was selected that showed a digestion pattern as expected for an insert extending from the BamHI site in Ad5 to the right ITR.

Sequence analysis of the cloning border at the right ITR revealed that the most 3′ G residue of the ITR was missing, the remainder of the ITR was found to be correct. Said missing G residue is complemented by the other ITR during replication.

2. TBr/Ad.Sal-rITR (ECACC Deposit P97082119)

pBr/Ad.Bam-rITR was digested with BamHI and SalI. The vector fragment including the adenovirus insert was isolated in LMP agarose (SeaPlaque GTG) and ligated to a 4.8 kb SalI-BamHI fragment obtained from wt Ad5 DNA and purified with the Geneclean II kit (Bio 101, Inc.). One clone was chosen and the integrity of the Ad5 sequences was determined by restriction enzyme analysis. Clone pBr/Ad.Sal-rITR contains adeno type 5 sequences from the SalI site at bp 16746 up to and including the rITR (missing the most 3′ G residue).

3. pBr/Ad.Cla-Bam (ECACC Deposit P97082117)

wt Adeno type 5 DNA was digested with ClaI and BamHI, and the 20.6 kb fragment was isolated from gel by electro-elution. pBr322 was digested with the same enzymes and purified from agarose gel by Geneclean. Both fragments were ligated and transformed into competent DH5a. The resulting clone pBr/Ad.Cla-Bam was analysed by restriction enzyme digestion and shown to contain an insert with adenovirus sequences from bp 919 to 21566.

4. pBr/Ad.AflII-Bam (ECACC Deposit P97082114)

Clone pBr/Ad.Cla-Bam was linearised with EcoRI (in pBr322) and partially digested with AflII. After heat inactivation of AflII for 20′ at 65° C. the fragment ends were filled in with Klenow enzyme. The DNA was then ligated to a blunt double stranded oligo linker containing a PacI site (5′-AATTGTCTTAATTAACCGCTTAA-3′). This linker was made by annealing the following two oligonucleotides: 5′-AATTGTCTTAATTAACCGC-3′ and 5′-AATTGCGGTTAATTAAGAC-3′, followed by blunting with Klenow enzyme. After precipitation of the ligated DNA to change buffer, the ligations were digested with an excess PacI enzyme to remove concatameres of the oligo. The 22016 bp partial fragment containing Ad5 sequences from bp 3534 up to 21566 and the vector sequences, was isolated in LMP agarose (SeaPlaque GTG), religated and transformed into competent DH5a. One clone that was found to contain the PacI site and that had retained the large adeno fragment was selected and sequenced at the 5′ end to verify correct insertion of the PacI linker in the (lost) AflII site.

5. pBr/Ad.Bam-rITRpac#2 (ECACC Deposit P97082120) and pBr/Ad.Bam-rITR#8 (ECACC Deposit P97082121)

To allow insertion of a PacI site near the ITR of Ad5 in clone pBr/Ad.Bam-rITR about 190 nucleotides were removed between the ClaI site in the pBr322 backbone and the start of the ITR sequences. This was done as follows: pBr/Ad.Bam-rITR was digested with ClaI and treated with nuclease Bal31 for varying lengths of time (2′, 5′, 10′ and 15′). The extent of nucleotide removal was followed by separate reactions on pBr322 DNA (also digested at the ClaI site), using identical buffers and conditions. Bal31 enzyme was inactivated by incubation at 75° C. for 10 minutes, the DNA was precipitated and resuspended in a smaller volume of TE buffer. To ensure blunt ends, DNAs were further treated with T4 DNA polymerase in the presence of excess dNTPs. After digestion of the (control) pBr322 DNA with SalI, satisfactory degradation (−150 bp) was observed in the samples treated for 10′ or 15′. The 10′ or 15′ treated pBr/Ad.Bam-rITR samples were then ligated to the above described blunted PacI linkers (See pBr/Ad.AflII-Bam). Ligations were purified by precipitation, digested with excess PacI and separated from the linkers on an LMP agarose gel. After religation, DNAs were transformed into competent DH5a and colonies analysed. Ten clones were selected that showed a deletion of approximately the desired length and these were further analysed by T-track sequencing (T7 sequencing kit, Pharmacia Biotech). Two clones were found with the PacI linker inserted just downstream of the rITR. After digestion with PacI, clone #2 has 28 bp and clone #8 has 27 bp attached to the ITR.

pWE/Ad.AflII-rITR (ECACC Deposit P97082116)

Cosmid vector pWE15 (Clontech) was used to clone larger Ad5 inserts. First, a linker containing a unique PacI site was inserted in the EcoRI sites of pWE15 creating pWE.pac. To this end, the double stranded PacI oligo as described for pBr/Ad.AflII-BamHI was used but now with its EcoRI protruding ends. The following fragments were then isolated by electro-elution from agarose gel: pWE.pac digested with PacI, pbr/AflII-Bam digested with PacI and BamHI and pBr/Ad.Bam-rITR#2 digested with BamHI and PacI. These fragments were ligated together and packaged using 1 phage packaging extracts (Stratagene) according to the manufacturers protocol. After infection into host bacteria, colonies were grown on plates and analysed for presence of the complete insert. pWE/Ad.AflII-rITR contains all adenovirus type 5 sequences from bp 3534 (AflII site) up to and including the right ITR (missing the most 3′ G residue).

pBr/Ad.lITR-Sal(9.4) (ECACC Deposit P97082115)

Adeno 5 wt DNA was treated with Klenow enzyme in the presence of excess dNTPs and subsequently digested with SalI. Two of the resulting fragments, designated left ITR-Sal(9.4) and Sal(16.7)-right ITR, respectively, were isolated in LMP agarose (Seaplaque GTG). pBr322 DNA was digested with EcoRV and SalI and treated with phosphatase (Life Technologies). The vector fragment was isolated using the Geneclean method (BIO 101, Inc.) and ligated to the Ad5 SalI fragments. Only the ligation with the 9.4 kb fragment gave colonies with an insert. After analysis and sequencing of the cloning border a clone was chosen that contained the full ITR sequence and extended to the SailI site at bp 9462.

pBr/Ad.lITR-Sal(16.7) (ECACC Deposit P97082118)

pBr/Ad.lITR-Sal(9.4) is digested with SalI and dephosphorylated (TSAP, Life Technologies). To extend this clone up to the third SalI site in Ad5, pBr/Ad.Cla-Bam was linearised with BamHI and partially digested with SalI. A 7.3 kb SalI fragment containing adenovirus sequences from 9462-16746 was isolated in LMP agarose gel and ligated to the SalI-digested pBr/Ad.lITR-Sal(9.4) vector fragment.

pWE/Ad.AflII-EcoRI

pWE.pac was digested with ClaI and 5′ protruding ends were filled using Klenow enzyme. The DNA was then digested with PacI and isolated from agarose gel. pWE/AflII-rITR was digested with EcoRI and after treatment with Klenow enzyme digested with PacI. The large 24 kb fragment containing the adenoviral sequences was isolated from agarose gel and ligated to the ClaI-digested and blunted pWE.pac vector using the Ligation Express™ kit from Clontech. After transformation of Ultracompetent XL10-Gold cells from Stratagene, clones were identified that contained the expected insert. pWE/AflII-EcoRI contains Ad5 sequences from bp 3534-27336.

Construction of New Adapter Plasmids

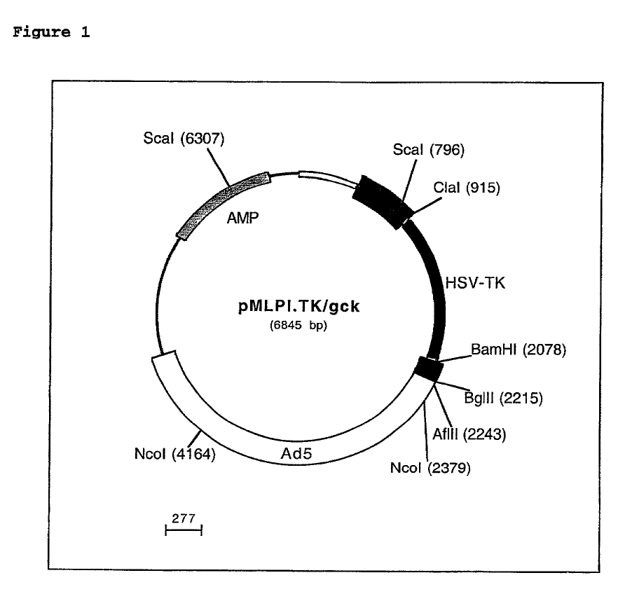

The absence of sequence overlap between the recombinant adenovirus and E1 sequences in the packaging cell line is essential for safe, RCA-free generation and propagation of new recombinant viruses. The adapter plasmid PMLPI.TK (FIG. 1) is an example of an adapter plasmid designed for use according to the invention in combination with the improved packaging cell lines of the invention. This plasmid was used as the starting material to make a new vector in which nucleic acid molecules comprising specific promoter and gene sequences can be easily exchanged.

First, a PCR fragment was generated from pZipΔMo+PyF101(N⁻) template DNA (described in PCT/NL96/00195) with the following primers: LTR-1: 5′-CTG TAC GTA CCA GTG CAC TGG CCT AGG CAT GGA AAA ATA CAT AAC TG-3′ and LTR-2: 5′-GCG GAT CCT TCG AAC CAT GGT AAG CTT GGT ACC GCT AGC GTT AAC CGG GCG ACT CAG TCA ATC G-3′. Pwo DNA polymerase (Boehringer Mannheim) was used according to manufacturers protocol with the following temperature cycles: once 5′ at 95° C.; 3′ at 55° C.; and 1′ at 72° C., and 30 cycles of 1′ at 95° C., 1′ at 60° C., 1′ at 72° C., followed by once 10′ at 72° C. The PCR product was then digested with BamHI and ligated into pMLP10 (Levrero et al., 1991) vector digested with PvuII and BamHI, thereby generating vector pLTR10. This vector contains adenoviral sequences from bp 1 up to bp 454 followed by a promoter consisting of a part of the Mo-MuLV LTR having its wild-type enhancer sequences replaced by the enhancer from a mutant polyoma virus (PyF101). The promoter fragment was designated L420. Next, the coding region of the murine HSA gene was inserted. pLTR10 was digested with BstBI followed by Klenow treatment and digestion with NcoI. The HSA gene was obtained by PCR amplification on pUC18-HSA (Kay et al., 1990) using the following primers: HSA1, 5′-GCG CCA CCA TGG GCA GAG CGA TGG TGG C-3′ and HSA2, 5′-GTT AGA TCT AAG CTT GTC GAC ATC GAT CTA CTA ACA GTA GAG ATG TAG AA-3′. The 269 bp amplified fragment was subcloned in a shuttle vector using the NcoI and BglII sites. Sequencing confirmed incorporation of the correct coding sequence of the HSA gene, but with an extra TAG insertion directly following the TAG stop codon. The coding region of the HSA gene, including the TAG duplication was then excised as a NcoI (sticky)-SalI (blunt) fragment and cloned into the 3.5 kb NcoI(sticky)/BstBI(blunt) fragment from pLTR10, resulting in pLTR-HSA10.

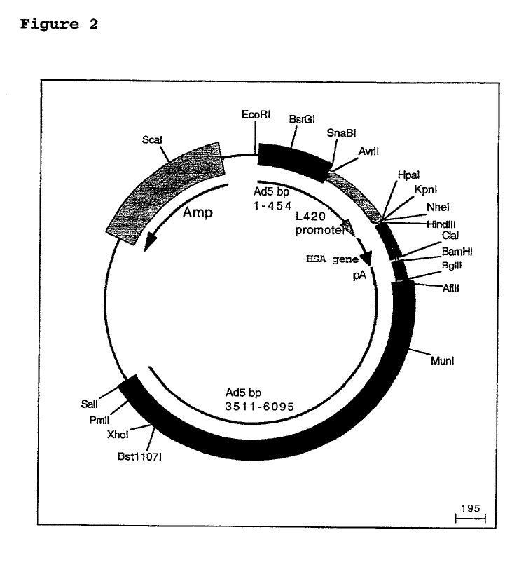

Finally, pLTR-HSA10 was digested with EcoRI and BamHI after which the fragment containing the left ITR, packaging signal, L420 promoter and HSA gene was inserted into vector PMLPI.TK digested with the same enzymes and thereby replacing the promoter and gene sequences. This resulted in the new adapter plasmid pAd/L420-HSA (FIG. 2) that contains convenient recognition sites for various restriction enzymes around the promoter and gene sequences. SnaBI and AvrII can be combined with HpaI, NheI, KpnI, HindIII to exchange promoter sequences, while the latter sites can be combined with the ClaI or BamHI sites 3′ from HSA coding region to replace genes in this construct.

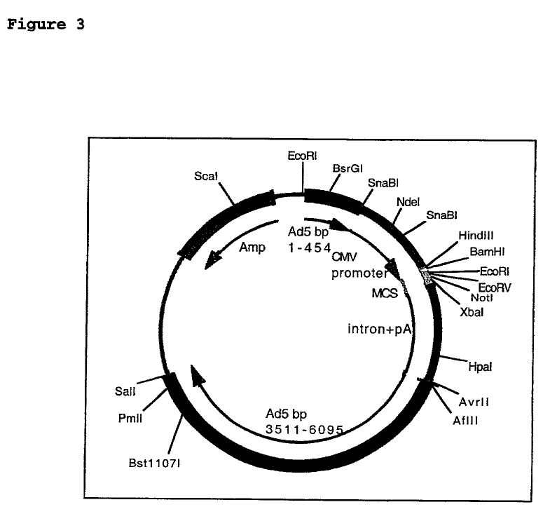

Another adapter plasmid that was designed to allow easy exchange of nucleic acid molecules was made by replacing the promoter, gene and poly A sequences in pAd/L420-HSA with the CMV promoter, a multiple cloning site, an intron and a poly-A signal. For this purpose, pAd/L420-HSA was digested with AvrII and BglII followed by treatment with Klenow to obtain blunt ends. The 5.1 kb fragment with pBr322 vector and adenoviral sequences was isolated and ligated to a blunt 1570 bp fragment from pcDNA1/amp (Invitrogen) obtained by digestion with HhaI and AvrII followed by treatment with T4 DNA polymerase. This adapter plasmid was named pCLIP (FIG. 3).

Generation of Recombinant Adenoviruses

To generate E1 deleted recombinant adenoviruses with the new plasmid-based system, the following constructs are prepared:

a) An adapter construct containing the expression cassette with the gene of interest linearised with a restriction enzyme that cuts at the 3′ side of the overlapping adenoviral genome fragment, preferably not containing any pBr322 vector sequences, and

b) A complementing adenoviral genome construct pWE/Ad.AflII-rITR digested with PacI.

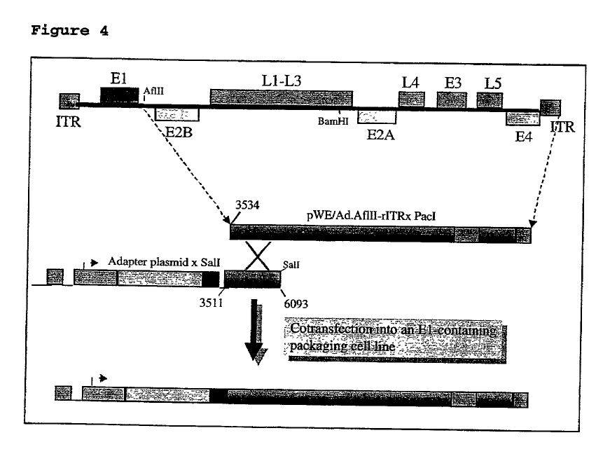

These two DNA molecules are further purified by phenol/chloroform extraction and EtOH precipitation. Co-transfection of these plasmids into an adenovirus packaging cell line, preferably a cell line according to the invention, generates recombinant replication deficient adenoviruses by a one-step homologous recombination between the adapter and the complementing construct (FIG. 4).

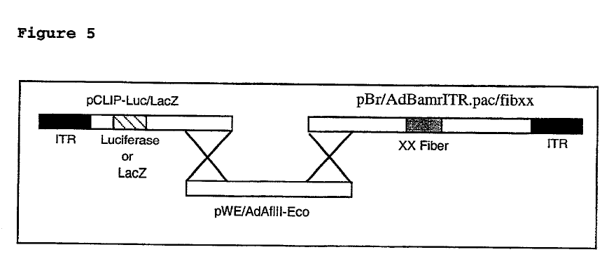

Alternatively, in stead of pWE/Ad.AflII-rITR other fragments can be used, e.g., pBr/Ad.Cla-Bam digested with EcoRI and BamHI or pBr/Ad.AflII-BamHI digested with PacI and BamHI can be combined with pBr/Ad.Sal-rITR digested with SalI. In this case, three plasmids are combined and two homologous recombinations are needed to obtain a recombinant adenovirus (FIG. 5). It is to be understood that those skilled in the art may use other combinations of adapter and complementing plasmids without departing from the present invention.

A general protocol as outlined below and meant as a non-limiting example of the present invention has been performed to produce several recombinant adenoviruses using various adapter plasmids and the Ad.AflII-rITR fragment. Adenovirus packaging cells (PER.C6) were seeded in ^{˜}25 cm² flasks and the next day when they were at ^{˜}80% confluency, transfected with a mixture of DNA and lipofectamine agent (Life Techn.) as described by the manufacturer. Routinely, 40 μl lipofectamine, 4 μg adapter plasmid and 4 μg of the complementing adenovirus genome fragment AflII-rITR (or 2 μg of all three plasmids for the double homologous recombination) are used. Under these conditions transient transfection efficiencies of ^{˜}50% (48 hrs post transfection) are obtained as determined with control transfections using a pAd/CMV-LacZ adapter. Two days later, cells are passaged to ^{˜}80 cm² flasks and further cultured. Approximately five (for the single homologous recombination) to eleven days (for the double homologous recombination) later a cytopathogenic effect (CPE) is seen, indicating that functional adenovirus has formed. Cells and medium are harvested upon full CPE and recombinant virus is released by freeze-thawing. An extra amplification step in an 80 cm² flask is routinely performed to increase the yield since at the initial stage the titers are found to be variable despite the occurrence of full CPE. After amplification, viruses are harvested and plaque purified on PER.C6 cells. Individual plaques are tested for viruses with active transgenes.

Besides replacements in the E1 region it is possible to delete or replace (part of) the E3 region in the adenovirus because E3 functions are not necessary for the replication, packaging and infection of the (recombinant) virus. This creates the opportunity to use a larger insert or to insert more than one gene without exceeding the maximum package size (approximately 105% of wt genome length). This can be done, e.g., by deleting part of the E3 region in the pBr/Ad.Bam-rITR clone by digestion with XbaI and religation. This removes Ad5 wt sequences 28592-30470 including all known E3 coding regions. Another example is the precise replacement of the coding region of gp19K in the E3 region with a polylinker allowing insertion of new sequences. This, 1) leaves all other coding regions intact and 2) obviates the need for a heterologous promoter since the transgene is driven by the E3 promoter and pA sequences, leaving more space for coding sequences.

To this end, the 2.7 kb EcoRI fragment from wt Ad5 containing the 5′ part of the E3 region was cloned into the EcoRI site of pBluescript (KS⁻) (Stratagene). Next, the HindIII site in the polylinker was removed by digestion with EcoRV and HincII and subsequent religation. The resulting clone pBS.Eco-Eco/ad5DHIII was used to delete the gp19K coding region. Primers 1 (5′-GGG TAT TAG GCC AA AGG CGC A-3′) and 2 (5′-GAT CCC ATG GAA GCT TGG GTG GCG ACC CCA GCG-3′) were used to amplify a sequence from pBS.Eco-Eco/Ad5DHIII corresponding to sequences 28511 to 28734 in wt Ad5 DNA. Primers 3 (5′-GAT CCC ATG GGG ATC CTT TAC TAA GTT ACA AAG CTA-3′) and 4 (5′-GTC GCT GTA GTT GGA CTG G-3′) were used on the same DNA to amplify Ad5 sequences from 29217 to 29476. The two resulting PCR fragments were ligated together by virtue of the new introduced NcoI site and subsequently digested with XbaI and MunI. This fragment was then ligated into the pBS.Eco-Eco/ad5 AHIII vector that was digested with XbaI (partially) and MunI generating pBS.Eco-Eco/ad5ΔHIII.Δgp19K. To allow insertion of foreign genes into the HindIII and BamHI site, an XbaI deletion was made in pBS.Eco-Eco/ad5 ΔRIII.Δgp19K to remove the BamHI site in the Bluescript polylinker The resulting plasmid pBS.Eco-Eco/ad5ΔHIIIΔgp19KΔXbaI, contains unique HindIII and BamHI sites corresponding to sequences 28733 (HindIII) and 29218 (BamHI) in Ad5. After introduction of a foreign gene into these sites, either the deleted XbaI fragment is re-introduced, or the insert is recloned into pBS.Eco-Eco/ad5ΔHIII.Δgp19K using HindIII and for example MunI. Using this procedure, we have generated plasmids expressing HSV-TK, hIL-1a, rat IL-3, luciferase or LacZ. The unique SrfI and NotI sites in the pBS.Eco-Eco/ad5ΔHIII.Δgp19K plasmid (with or without inserted gene of interest) are used to transfer the region comprising the gene of interest into the corresponding region of pBr/Ad.Bam-rITR, yielding construct pBr/Ad.Bam-rITRΔgp19K (with or without inserted gene of interest). This construct is used as described supra to produce recombinant adenoviruses. In the viral context, expression of inserted genes is driven by the adenovirus E3 promoter.

Recombinant viruses that are both E1 and E3 deleted are generated by a double homologous recombination procedure as described above for E1-replacement vectors using a plasmid-based system consisting of:

a) an adapter plasmid for E1 replacement according to the invention, with or without insertion of a first gene of interest,

b) the pWE/Ad.AflII-EcoRI fragment, and

c) the pBr/Ad.Bam-rITRΔgp19K plasmid with or without insertion of a second gene of interest.

In addition to manipulations in the E3 region, changes of (parts of) the E4 region can be accomplished easily in pBr/Ad.Bam-rITR. Generation and propagation of such a virus, however, in some cases demands complementation in trans.

### EXAMPLE 2

### Generation of Adenovirus Serotype 5 Based Viruses with Chimaeric Fiber Proteins

The method described infra to generate recombinant adenoviruses by co-transfection of two, or more separate cloned adenovirus sequences. One of these cloned adenovirus sequences was modified such that the adenovirus serotype 5 fiber DNA was deleted and substituted for unique restriction sites thereby generating ìtemplate clonesî which allow for the easy introduction of DNA sequences encoding for fiber protein derived from other adenovirus serotypes.

Generation of Adenovirus Template Clones Lacking DNA Encoding for Fiber

The fiber coding sequence of adenovirus serotype 5 is located between nucleotides 31042 and 32787. To remove the adenovirus serotype 5 DNA encoding fiber we started with construct pBr/Ad.Sam-rITR. First a NdeI site was removed from this construct. For this purpose, pBr322 plasmid DNA was digested with NdeI after which protruding ends were filled using Klenow enzyme. This pBr322 plasmid was then re-ligated, digested with NdeI and transformed into E. coli DH5α. The obtained pBr/ΔNdeI plasmid was digested with ScaI and SalI and the resulting 3198 bp vector fragment was ligated to the 15349 bp ScaI-SalI fragment derived from pBr/Ad.BamrITR, resulting in plasmid pBr/Ad.Bam-rITRΔNdeI which hence contained a unique NdeI site. Next a PCR was performed with oligonucleotides NY-up: 5′-CGA CAT ATG TAG ATG CAT TAG TTT GTG TTA TGT TTC AAC GTG-3′

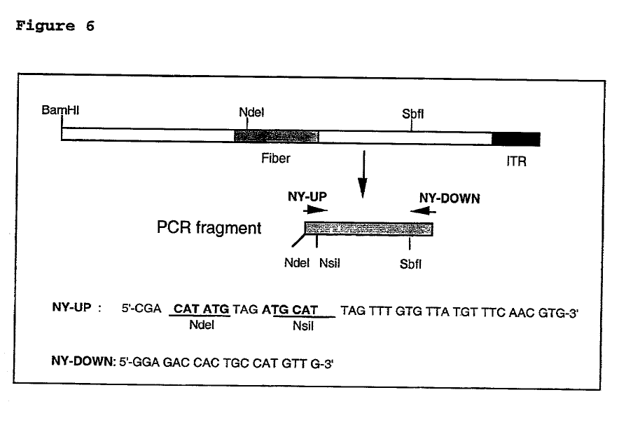

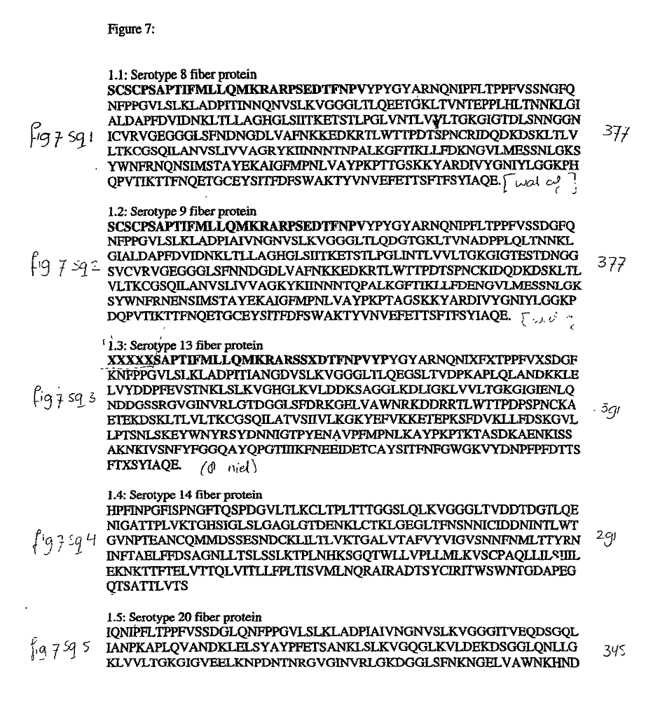

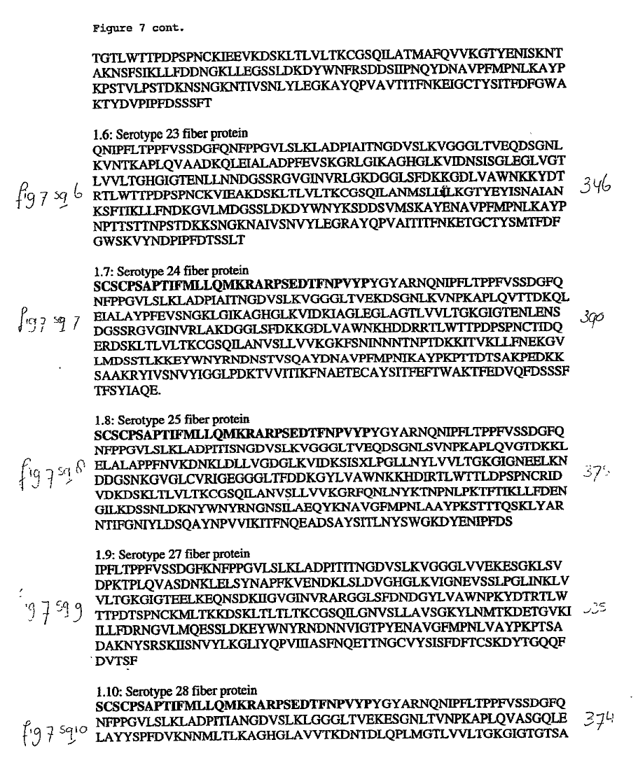

And NY-down:5′-GGA GAC CAC TGC CAT GTT-3′ (FIG. 6). During amplification, both a NdeI (bold face) and a NsiI restriction site (underlined) were introduced to facilitate cloning of the amplified fiber DNAs. Amplification consisted of 25 cycles of each 45 sec. at 94° C., 1 min. at 60° C., and 45 sec. at 72° C. The PCR reaction contained 25 pmol of oligonucleotides NY-up or NY-down, 2mM DNTP, PCR buffer with 1.5 mM MgCl₂, and 1 unit of Elongase heat stable polymerase (Gibco, The Netherlands). One-tenth of the PCR product was run on an agarose gel which demonstrated that the expected DNA fragment of ±2200 bp was amplified. This PCR fragment was subsequently purified using Geneclean kit system (Bio101 Inc.). Then, both the construct pBr/Ad.Bam-rITRΔNdeI as well as the PCR product were digested with restriction enzymes NdeI and SbfI. The PCR fragment was subsequently cloned using T4 ligase enzyme into the NdeI and SbfI digested pBr/Ad.Bam-rITRΔNdeI, generating pBr/Ad.BamRΔFib. This plasmid allows insertion of any PCR amplified fiber sequence through the unique NdeI and NsiI sites that are inserted in place of the removed fiber sequence. Viruses can be generated by a double homologous recombination in packaging cells described infra using an adapter plasmid, construct pBr/Ad.AflII-EcoRI digested with PacI and EcoRI and a pBr/Ad.BamRΔFib construct in which heterologous fiber sequences have been inserted. To increase the efficiency of virus generation, the construct pBr/Ad.BamRΔFib was modified to generate a PacI site flanking the right ITR. Hereto, pBr/Ad.BamRΔFib was digested with AvrII and the 5 kb adeno fragment was isolated and introduced into the vector pBr/Ad.Bam-rITR.pac#8 replacing the corresponding AvrII fragment. The resulting construct was named pBr/Ad.BamRΔFib.pac. Once a heterologous fiber sequence is introduced in pBr/Ad.BamRΔFib.pac, the fiber modified right hand adenovirus clone may be introduced into a large cosmid clone as described for pWE/Ad.AflII-rITR in example 1. Such a large cosmid clone allows generation of adenovirus by only one homologous recombination making the process extremely efficient.

Amplification of Fiber Sequences from Adenovirus Serotvpes

To enable amplification of the DNAs encoding fiber protein derived from alternative serotypes degenerate oligonucleotides were synthesised. For this purpose, first known DNA sequences encoding for fiber protein of alternative serotypes were aligned to identify conserved regions in both the tail-region as well as the knob-region of the fiber protein. From the alignment, which contained the nucleotide sequence of 19 different serotypes representing all 6 subgroups, (degenerate) oligonucleotides were synthesised (see table 2). Also shown in table 3 is the combination of oligonucleotides used to amplify the DNA encoding fiber protein of a specific serotype. The amplification reaction (50 μl) contained 2 mM dNTPs, 25 pmol of each oligonucleotide, standard 1× PCR buffer, 1,5 mM MgCl₂, and 1 Unit Pwo heat stable polymerase (Boehringer) per reaction. The cycler program contained 20 cycles, each consisting of 30 sec. 94°C., 60 sec. 60-64° C., and 120 sec. At 72° C. One-tenth of the PCR product was run on an agarose gel which demonstrated that a DNA fragment was amplified. Of each different template, two independent PCR reactions were performed after which the independent PCR fragments obtained were sequenced to determine the nucleotide sequence. From 11 different serotypes, the nucleotide sequence could be compared to sequences present in Genbank. Of all other serotypes, the DNA encoding fiber protein was previously unknown and was therefore aligned with known sequences from other subgroup members to determine homology i.e. sequence divergence. of the 51 human serotypes known to date, all fiber sequences, except for serotypes 1, 6, and 26, have been amplified and sequenced. The protein sequences of the fiber from different adenovirus serotypes is given in FIG. 7.

Generation of Fiber Chimaeric Adenoviral DNA Constructs

All amplified fiber DNAs as well as the vector (pBr/Ad.BamRΔFib) were digested with NdeI and NsiI. The digested DNAs was subsequently run on a agarose gel after which the fragments were isolated from the gel and purified using the Geneclean kit (Bio101 Inc). The PCR fragments were then cloned into the NdeI and NsiI sites of pBr/AdBamRΔFib, thus generating pBr/AdBamRFibXX (where XX stands for the serotype number of which the fiber DNA was isolated). So far the fiber sequence of serotypes 5/7/8/9/10/11/12/13/14/16/17/19/21/24/27/28/29/30/32/33/34/35/36/37/38/40-S/40-L/41-S/42/45/47/49/51 have been cloned into pBr/AdBamRFibXX. From pBr/AdBamRFibXX (where XX is 5/8/9/10/11/13/16/17/24/27/30/32/33/34/35/38/40-S/40-L/45/47/49/51) an 6 kb AvrII fragment encompassing the fiber sequence was isolated via gelelectrophoresis and Geneclean. This AvrII fragment was subsequently cloned in plasmid pBr/Ad.Bam-rITR.pac (see example 1) which was digested to completion with AvrII and dephosphorylated as described previously, leading to the generation of the plasmid pBr/Ad.Bam-rITR.pac.fibXX. This plasmid was subsequently used to generate a cosmid clone with a modified fiber using the constructs pWE.pac, pBr/AflII-Bam and pBr/Ad.Bam-rITR.pac.fibXX. This cosmid cloning resulted in the formation of construct pWE/Ad.AflII-rITR/FibXX (where XX stands for the serotype number of which the fiber DNA was isolated).

Generation of pAd5/L420.HSA, pAd5/Clip and pAd5/Clipsal

pMLPI.TK was used to make a new vector in which nucleic acid molecules comprising specific promoter and gene sequences can be easily exchanged.

First, a PCR fragment was generated from pZipΔMo+PyF101(N⁻) template DNA (described in PCT/NL96/00195) with the following primers: LTR-1: 5′-CTG TAC GTA CCA GTG CAC TGG CCT AGG CAT GGA AAA ATA CAT AAC TG-3′ and LTR-2: 5′-GCG GAT CCT TCG AAC CAT GGT AAG CTT GGT ACC GCT AGC GTT AAC CGG GCG ACT CAG TCA ATC G-3′. Pwo DNA polymerase (Boehringer Mannheim) was used according to manufacturers protocol with the following temperature cycles: once 5′ at 95° C.; 3′ at 55° C.; and 1′ at 72° C., and 30 cycles of 1′ at 95° C., 11 at 60° C., 1′ at 72° C., followed by once 10′ at 72° C. The PCR product was then digested with BamHI and ligated into pMLP10 (Levrero et al., 1991; Gene 101, 195-202) digested with PvuII and BamHI, thereby generating vector pLTR10. This vector contains adenoviral sequences from bp 1 up to bp 454 followed by a promoter consisting of a part of the Mo-MuLV LTR having its wild-type enhancer sequences replaced by the enhancer from a mutant polyoma virus (PyF101). The promoter fragment was designated L420. Sequencing confirmed correct amplification of the LTR fragment however the most 5′ bases in the PCR fragment were missing so that the PvuII site was not restored. Next, the coding region of the murine HSA gene was inserted. pLTR10was digested with BstBI followed by Klenow treatment and digestion with NcoI. The HSA gene was obtained by PCR amplification on pUC18-HSA (Kay et al., 1990; J. Immunol. 145, 1952-1959) using the following primers: HSA1, 5′-GCG CCA CCA TGG GCA GAG CGA TGG TGG C-3′ and HSA2, 5′-GTT AGA TCT AAG CTT GTC GAC ATC GAT CTA CTA ACA GTA GAG ATG TAG AA-3′. The 269 bp amplified fragment was subcloned in a shuttle vector using the NcoI and BglII sites. Sequencing confirmed incorporation of the correct coding sequence of the HSA gene, but with an extra TAG insertion directly following the TAG stop codon. The coding region of the HSA gene, including the TAG duplication was then excised as a NcoI(sticky)-SalI(blunt) fragment and cloned into the 3.5 kb NcoI(sticky)/BstBI(blunt) fragment from pLTR10, resulting in pLTR-HSA10.

Finally, pLTR-HSA10 was digested with EcoRI and BamHI after which the fragment containing the left ITR, packaging signal, L420 promoter and HSA gene was inserted into vector pMLPI.TK digested with the same enzymes and thereby replacing the promoter and gene sequences. This resulted in the new adapter plasmid pAd5/L420-HSA that contains convenient recognition sites for various restriction enzymes around the promoter and gene sequences. SnaBI and AvrII can be combined with HpaI, NheI, KpnI, HindIII to exchange promoter sequences, while the latter sites can be combined with the ClaI or BamHI sites 3′ from HSA coding region to replace genes in this construct.

Another adapter plasmid that was designed to allow easy exchange of nucleic acid molecules was made by replacing the promoter, gene and polyA sequences in pAd5/L420-HSA with the CMV promoter, a multiple cloning site, an intron and a polyA signal. For this purpose, pAd5/L420-HSA was digested with AvrII and BglII followed by treatment with Klenow to obtain blunt ends. The 5.1 kb fragment with pBr322 vector and adenoviral sequences was isolated and ligated to a blunt 1570 bp fragment from pcDNA1/amp (Invitrogen) obtained by digestion with HhaI and AvrII followed by treatment with T4 DNA polymerase. This adapter plasmid was named pAd5/Clip. To enable removal of vector sequences from the adenoviral fragment pAd5/Clip was partially digested with EcoRI and the linear fragment was isolated. An oligo of the sequence 5′ TTAAGTCGAC-3′ was annealed to itself resulting in a linker with a SalI site and EcoRI overhang. The linker was ligated to the partially digested pAd5/Clip vector and clones were selected that had the linker inserted in the EcoRI site 23 bp upstream of the left adenovirus ITR in pAd5/Clip resulting in pAd5/Clipsal.

Generation of pAd5ClipLacZ, TAd5Clip.Luc, pAd5Clip.TK and pAd5Clipsal.Luc

The adapter plasmid pAd5/Clip.LacZ was generated as follows: The E. coli LacZ gene was amplified from the plasmid pMLP.nlsLacZ (EP 95-202 213) by PCR with the primers 5′ GGGGTGGCCAGGGTACCTCTAGGCTTTTGCAA and 5′ GGGGGGATCCATAAACAAGTTCAGAATCC. The PCR reaction was performed Ex Taq (Takara) according to the suppliers protocol at the following amplification program: 5 minutes 94° C., 1 cycle; 45 seconds 94° C. and 30 seconds 60° C and 2 minutes 72° C., 5 cycles; 45 seconds 94° C. and 30 seconds 65° C. and 2 minutes 72° C., 25 cycles; 10 minutes 72; 45 seconds 94° C. and 30 seconds 60° C. and 2 minutes 72° C., 5 cycles, 1 cycle. The PCR product was subsequently digested with Kpn1 and BamHI and the digested DNA fragment was ligated into Kpn1/BamHI digested pcDNA3 (Invitrogen), giving rise to pcDNA3.nlsLacZ. Next, the plasmid pAd5/Clip was digested with SpeI. The large fragment containing part of the 5′ part CMV promoter and the adenoviral sequences was isolated. The plasmid pcDNA3.nlsLacZ was digested with SpeI and the fragment containing the 3′ part of the CMV promoter and the lacz gene was isolated. Subsequently, the fragments were ligated, giving rise to pAd/Clip.LacZ. The reconstitution of the CMV promoter was confirmed by restriction digestion.

The adapter plasmid pAd5/Clip.Luc was generated as follows: The plasmid pCMV.Luc (EP 95-202 213) was digested with HindIII and BamHI. The DNA fragment containing the luciferase gene was isolated. The adapter plasmid pAd5/Clip was digested with HindIII and BamHI, and the large fragment was isolated. Next, the isolated DNA fragments were ligated, giving rise to pAd5/Clip.Luc. The adapter pClipsal.Luc was generated in the same way but using the adapter pClipsal digested with HIII and BamHI as vector fragment. Likewise, the TK containing HIII-BamHI fragment from pCMV.TK (EP 95-202 213) was inserted in pclipsal to generate pAd5/Clip.TK. The presence of the SalI site just upstream of the left ITR enables liberation of vector sequences from the adeno insert. Removal of these vector sequences enhances frequency of vector generation during homologous recombination in PER.C6.

Generation of Recombinant Adenovirus Chimaeric for Fiber Protein

To generate recombinant Ad 5 virus carrying the fiber of serotype 12, 16, 28, 40-L, 51, and 5, three constructs, pCLIP.Luc, pWE/AdAflII-Eco and pBr/AdBamrITR.pac/fibXX (XX=12, 16, 28, 40-L, 51, and 5) were transfected into adenovirus producer cells. To generate recombinant Ad 5 virus carrying the fiber of 5/7/8 9/10/11/12/13/14/16/17/19/21/24/27/28/29/30/32/33/34/35/36/37/38/40-S/40-L/41-S/42/45/47/49/51, two constructs pCLIP.Luc and pWE/Ad.AflII-rITR/FibXX were transfected into adenovirus producer cells.

For transfection, 2 4g of pCLIP.Luc, and 4 μg of both pWE/AdAflII-Eco and pBr/AdBamrITR.pac/fibXX (or in case of cosmids: 4 gg of pCLIP.Luc plus 4 μg of pWE/Ad.AflII-rITR/FibXX) were diluted in serum free DMEM to 100 μl total volume. To this DNA suspension 100 μl 1× diluted lipofectamine (Gibco) was added. After 30 minutes at room temperature the DNA-lipofectamine complex solution was added to 2.5 ml of serum-free DMEM which was subsequently added to a T25 cm² tissue culture flask. This flask contained 2×10⁶ PER.C6 cells that were seeded 24-hours prior to transfection. Two hours later, the DNA-lipofectamine complex containing medium was diluted once by the addition of 2.5 ml DMEM supplemented with 20% foetal calf serum. Again 24 hours later the medium was replaced by fresh DMEM supplemented with 10% foetal calf serum. Cells were cultured for 6-8 days, subsequently harvested, and freeze/thawed 3 times. Cellular debris was removed by centrifugation for 5 minutes at 3000 rpm room temperature. Of the supernatant (12.5 ml) 3-5 ml was used to infect again infect PER.C6 cells (T80 cm² tissue culture flasks). This re-infection results in full cytopathogenic effect (CPE) after 5-6 days after which the adenovirus is harvested as described above.

### EXAMPLE 3

### Production, Purification, and Titration of Fiber Chimaeric Adenoviruses

Of the supernatant obtained from transfected PER.C6 cells typically 10 ml was used to inoculate a 1 litre fermentor which contained 1-1.5×10⁶ cells/ml PER.C6 that were specifically adapted to grow in suspension. Three days after inoculation, the cells were harvested and pelleted by centrifuging for 10 min at 1750 rpm at room temperature. The chimaeric adenoviruses present in the pelleted cells were subsequently extracted and purified using the following downstream processing protocol. The pellet was dissolved in 50 ml 10 mM NaPO₄, and frozen at −20° C. After thawing at 37° C., 5.6 ml deoxycholate (5% w/v) was added after which the solution was homogenated. The solution was subsequently incubated for 15 minutes at 37° C. to completely crack the cells. After homogenising the solution, 1875 μl (1M) MgCl₂was added and 5 ml 100% glycerol. After the addition of 375 μl DNase (10 mg/ml) the solution was incubated for 30 minutes at 37° C. Cell debris was removed by centrifugation at 1880×g for 30 minutes at room temperature without the brake on. The supernatant was subsequently purified from proteins by loading on 10 ml of freon. Upon centrifugation for 15 minutes at 2000 rpm without brake at room temperature three bands are visible of which the upper band represents the adenovirus. This band was isolated by pipetting after which it was loaded on a Tris/HCl (1M) buffered caesiumchloride blockgradient (range: 1.2 to 1.4 gr./ml). Upon centrifugation at 21000 rpm for 2.5 hours at 10° C. the virus was purified from remaining protein and cell debris since the virus, in contrast to the other components, does not migrate into the 1.4 gr./ml caesiumchloride solution. The virus band is isolated after which a second purification using a Tris/HCl (1M) buffered continues gradient of 1.33 gr./ml of caesiumchloride is performed. After virus loading on top of this gradient the virus is centrifuged for 17 hours at 55000 rpm at 10° C. Subsequently the virus band is isolated and after the addition of 30 μl of sucrose (50 w/v) excess caesiumchloride is removed by three rounds of dialysis, each round comprising of 1 hour. For dialysis the virus is transferred to dialysis slides (Slide-a-lizer, cut off 10000 kDa, Pierce, USA). The buffers used for dialysis are PBS which are supplemented with an increasing concentration of sucrose (round 1 to 3:30 ml, 60 ml, and 150 ml sucrose (50% w/v)/ 1.5 litre PBS, all supplemented with 7.5 ml 2% (w/v) CaMgCl₂). After dialysis, the virus is removed from the slide-a-lizer after which it is aliquoted in portions of 25 and 100 μl upon which the virus is stored at −85° C.

To determine the number of virus particles per millilitre, 100 μl of the virus batch is run on an high pressure liquid chromatograph (HPLC). The adenovirus is bound to the column (anion exchange) after which it is eluted using a NaCl gradient (range 300-600 mM). By determining the area under the virus peak the number of virus particles can be calculated. To determine the number of infectious units (IU) per ml present in a virus batch, titrations are performed on 911 cells. For this purpose, 4×10⁴911 cells are seeded per well of 96-well plates in rows B, D, and F in a total volume of 100 μl per well. Three hours after seeding the cells are attached to the plastic support after which the medium can be removed. To the cells a volume of 200 μl is added, in duplicate, containing different dilutions of virus (range: 10² times diluted to 2×10⁹). By screening for CPE the highest virus dilution which still renders CPE after 14 days is considered to contain at least one infectious unit. Using this observation, together with the calculated amount of virus volume present in these wells renders the number of infectious units per ml of a given virus batch. The production results i.e. virus particles per ml and IU per ml or those chimaeric adenoviruses that were produced so far, are shown in table 3.

### EXAMPLE 4

### Presence of Ad5 Receptor Molecules on Human Cells

To investigate the importance of the presence of CAR on target cells for infection with chimaeric adenoviruses, a panel of human cell lines and primary cells were tested for the presence and/or absence of CAR, MHC class I, and integrins (αvβ3, αvβ5). For this purpose, 1×10⁵ target cells or were transferred to tubes (4 tubes per cell type) designed for flow cytometry. Cells were washed once with PBS/0.5% BSA after which the cells were pelleted by centrifugation for 5 minutes at 1750 rpm at room temperature. Subsequently, 10 μl of a 100 times diluted αᵥβ3 antibody (Mab 1961, Brunswick chemie, Amsterdam, The Netherlands), a 100 times diluted antibody αᵥβ5 (antibody (Mab 1976, Brunswick chemie, Amsterdam, The Netherlands), or 2000 times diluted CAR antibody was a kind gift of Dr. Bergelson, Harvard Medical School, Boston, USA (Hsu et al) was added to the cell pellet after which the cells were incubated for 30 minutes at 4° C. in a dark environment. After this incubation, cells were washed twice with PBS/0.5% BSA and again pelleted by centrifugation for 5 minutes at 1750 rpm room temperature. To label the cells, 10 μl of rat anti mouse IgG1 labelled with phycoerythrin (PE) was added to the cell pellet upon which the cells were again incubated for 30 minutes at 4° C. in a dark environment. Finally the cells were washed twice with PBS/0.5% BSA and analysed on a flow cytometer. The results of flow cytometric analysis of these experiments are shown in table 4. These results show that human erythroid leukemia cells (K562, ATCC: CCL-243), human primary fibroblasts (GM09503), human primary smooth muscle cells, and primary human synoviocytes do not express detectable levels of the CAR receptor. In contrast, human lung carcinoma cells (A549, ATCC: CCL-1185), human lymphoblast cells (SupT1 (B and T cell hybrid, ATCC, CRL-1991), and human liver cells (HEPG2, ATCC, HB8065) express high amounts of CAR protein. Human lymphoblast cells (CEM, ATCC: CRL-1992), primary human umbilical vein endothelial cells (HUVEC), and human primary chorion villi express low amounts of CAR protein.

### EXAMPLE 5

### Infection of CAR Negative Cells with Fiber Chimaeric Adenovirus

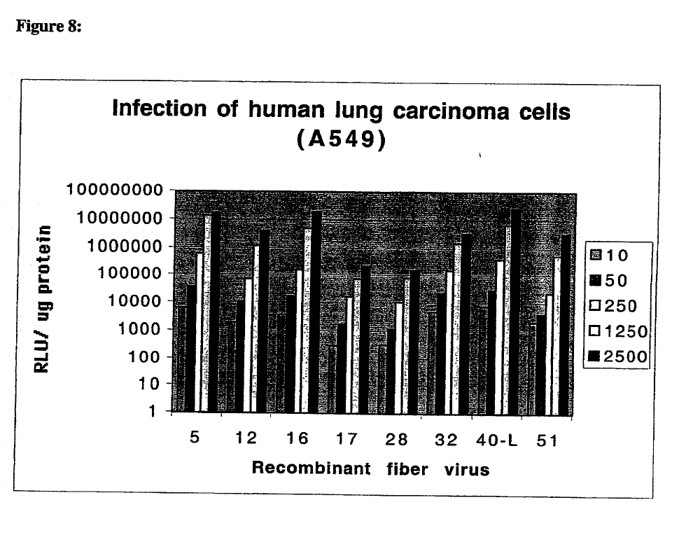

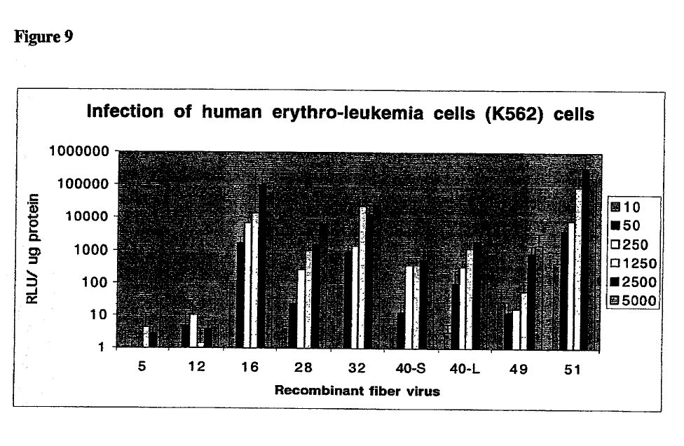

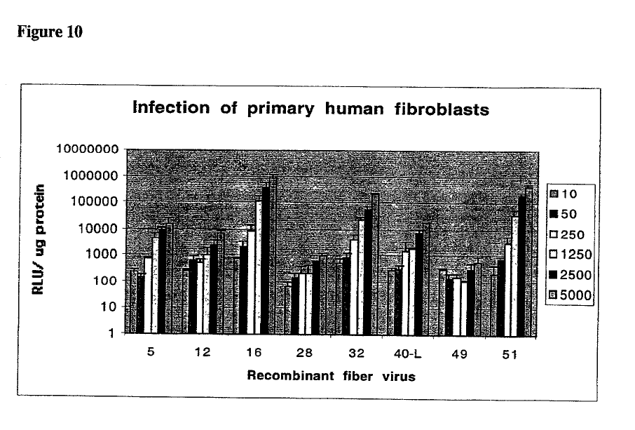

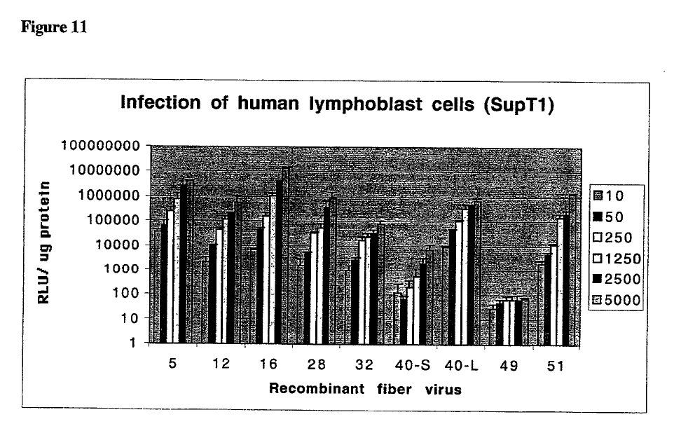

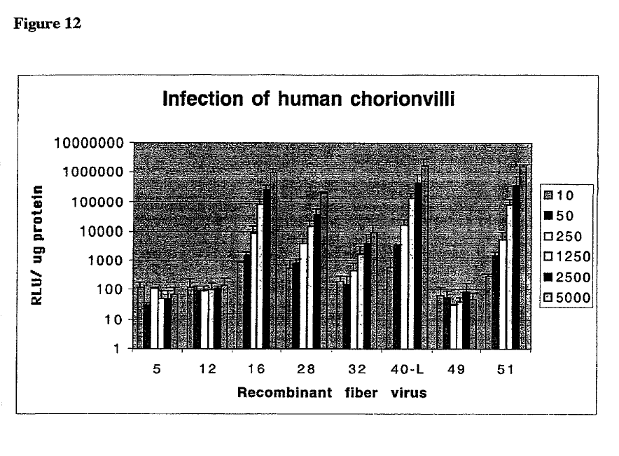

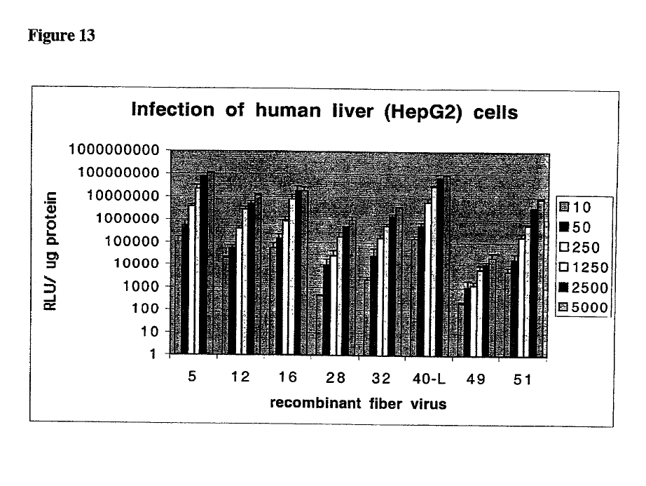

Several of the cell types described in example 4, i.e. A549, K562, GM09503, SupT1, chorion villi, and HepG2 were infected with a panel of chimaeric adenoviruses. This panel consists of adenovirus serotype 5 (subgroup C), and of adenovirus serotype 5 containing the fiber of serotypes 16 and 51 (subgroup B), of 28, 32, and 49 (subgroup D), of 12 (subgroup A), and of 40 (40-S and/or 40-L: subgroup F). For this purpose target cells are seeded at a concentration of 10 cells per well of 6-well plates in 2 ml Dulbecco's modified Eagles medium (DMEM, Life Technologies, The Netherlands) supplemented with 10% Foetal calf serum. Twenty-four hours later the medium is replaced by fresh medium containing the different chimaeric adenoviruses at an increasing MOI of 0, 10, 50, 250, 1250, 2500, 5000 (MOI based on virus particles per millilitre). Approximately 2 hours after the addition of virus the medium containing the virus is discarded, cells are washed once with PBS, and subsequently 2 ml of fresh medium (not containing virus) is added to each well. Forthy-eight hours later cells are harvested, washed and pelleted by centrifuging 5 minutes at 1500 rpm. Cells are subsequently lysed in 0,1 ml lysis buffer (1% Triton-X-100, 15% Glycerol, 2 mM EDTA, 2 mM DTT, and 25 mM MgCl₂ in Tris-phosphate buffer pH 7.8) after which the total protein concentration of the lysate is measured (Biorad, protein standard II). To determine marker gene expression (luciferase activity) 20 μl of the protein sample is mixed with 100 μl of a luciferase substrate (Luciferine, Promega, The Netherlands) and subsequently measured on a Lumat LB 9507 apparatus (EG & G Berthold, The Netherlands). The results of these infection experiments, given as the amount of luciferase activity (RLU) per μg protein, are shown in FIGS. 8-14. From these infection experiments several conclusions can be drawn. The infection of A549 cells (FIG. 8) demonstrates that all chimaeric adenoviruses tested infect with relative high efficiency these cells. The infection of K562 cells (FIG. 9) demonstrates that these cells cannot be transduced with adenovirus serotype 5 (subgroup C) or the fiber chimera 12 (subgroup A). All other chimaeric adenoviruses (16/ 51: subgroup B; 28/32/49: subgroup D; 40-L: subgroup F) are able to infect these cells with different efficiencies. The infection of GM09503 primary human fibroblasts (FIG. 10) demonstrates that these cells can be transduced with all fiber chimeras including Adenovirus serotype 5 albeit with different efficiencies. The infection of SupT1 cells (FIG. 11) demonstrates that these cells can be transduced with all fiber chimeras albeit with different efficiencies except for fiber chimera 49 which does not infect these human lymphoblast cells. The infection of human chorion villi cells (FIG. 12) shows a similar transduction pattern as observed with K562 cells except for adenovirus chimera 49 which does not infect these cells. The infection of HEPG2 cells (FIG. 13) shows a similar transduction pattern as observed with A549 cells. Linking the CAR expression data of these cells to the infection efficiency data obtained, several conclusions can be drawn. 1) Infection of adenovirus serotype 5 is correlated with the presence of CAR (FIG. 8-13). 2) In the absence of CAR but in the presence of high amount of MHC class I, poor infection is observed using adenovirus serotype 5, indicating that MHC class I is a worse receptor for adenovirus serotype 5 as compared to CAR (FIG. 10). 3) In the absence of CAR adenovirus fiber chimeras 16 and 51 (subgroup B) as well as chimeras 28 and 32 (subgroup D) as well as chimera 40-L (subgroup F) can infect cells with high efficiency, indicating that these viruses can utilise receptors other than CAR (FIGS. 9 and 12). 4) A comparison of the infection data of the chimaeric adenoviruses carrying the fiber of 28, 32, and 49 teaches that within an adenovirus subgroup differences in transduction efficiencies exist, indicating that adenovirus members of one subgroup either have different affinities for the same receptor, or that different adherence molecules can be used (FIGS. 8-13) by members of an adenovirus subgroup.

### EXAMPLE 6:

### Complexity of Receptor Recognition of Adenovirus Serotypes

To investigate the complexity and/or the number of different adherence molecules which can be used by human adenoviruses from different subgroups or between members within one subgroup the following strategies are designed.

1) Interference studies with total chimaeric viruses Via infection experiments described in example 5, cell lines are identified that are poorly transducible with a chimaeric viruses carrying the fiber protein of for example serotype 49 (subgroup D) indicating that such a cell expresses low levels of the adherence molecule required for D group adenovirus infection. Next, chimaeric adenoviruses carrying the fiber protein of other members of subgroup D are mixed in different concentrations with the fiber 49 chimaeric adenovirus and subsequently added to the cells. Since the fiber 49 chimaeric adenovirus carries a transgene other than the other subgroup D chimaeric adenoviruses (including but not limited to LacZ, Green Fluorescent Protein Yellow Fluorescent Protein, luciferase etc) interference of infection can be visualised. As a positive control two fiber 49 chimaeric adenoviruses carrying different marker genes is used. Identical to the example for subgroup D described above experiments are conducted with different members of subgroup A, B, C, E, and F. These experiments show if the fiber protein of members of the same adenovirus subgroup recognise the same adherence molecules on a cell membrane. Naturally, this approach is also used to investigate inter-subgroup variation for example usage of adherence molecules by subgroup D and B members

2) Interference Studies with Fiber Protein Derived Peptides

Peptides of 6-12 amino acids are synthetically synthesised which together form the complete knob domain of a fiber from a subgroup D, for example 49. Next, one or more peptides are mixed in various concentrations with the fiber 49 chimaeric adenovirus after which the mixture is added to the cells. Using this approach one or more peptides are identified which block, at a certain concentration, the infection of the fiber 49 chimaeric adenovirus. This peptide or these peptides are subsequently used to investigate whether the infection of other subgroup D members is blocked by addition of the peptide(s) and whether inhibition of infection occurs using the same concentration of peptide. Identical to the example for subgroup D described above peptides are synthesised using the knob domain of a member of subgroup A, B, C, E, and F. These experiments show not only which adherence molecules are used but also which part of the fiber protein is directly involved in binding to target cells. Naturally these peptides are also used to investigate inter-subgroup variation.

3) Interference Studies with Baculovirus Produced Recombinant Knob Proteins

Of each adenovirus subgroup, the knob region of one member is amplified by PCR. The forward oligonucleotide hybridises to the final repeat of the shaft part of the fiber just upstream of the start of the knob protein. This oligonucleotide contains a restriction site to facilitate cloning, a Histidine (6×) tag for purification after production, and a mutation thereby introducing a Methionine start codon. The reverse oligonucleotide hybridises after the polyA signal and contains a restriction site to facilitate cloning into a baculovirus expression construct. After generation of recombinant baculovirus, insect cells for instance Sf9, are infected. 4-6 days after infection cells are cracked by 3 cycles of freeze/thaw. Recombinant knob protein is purified from the supernatant using an antibody specifically recognising the His tag. The recombinant knobs are subsequently used in interference studies to investigate the complexity of adenovirus binding between members of different subgroups as well as members within one subgroup.

### EXAMPLE 7

### Identification of Adherence Molecules Involved in Adenovirus Subgroup B, D, and F Binding and Internalisation

To investigate what adherence molecules are involved in binding and internalisation of adenovirus serotypes from different subgroups in particular subgroups B, D, and F, the following strategies are designed.

1) Phage Display Libraries

Phage display libraries, containing random 6-12 amino acids peptides are ìmixedî with synthetically synthesised peptides which have identified to block infection of one or more members of either subgroup B, D, and/or F. Mixing of phages with peptide(s) is performed in an ELISA setting in which the peptide(s) are coated to a plastic support. Several rounds of mixing, washing and elution are performed to obtain an enrichment for phages that truly and specifically bind to the peptide(s). Finally the phages retrieved are amplified and plaque purified after which approximately 20 are sequenced to establish the nature of the peptide insert of the phages. From the consensus sequence of all 20 phages, a (degenerate) oligonucleotide is synthesised which together with a polyA hybridising oligonucleotide is used for the amplification of cDNA sequences both from cells which can or cannot (negative control) be infected with a subgroup B, D, and/or F chimaeric adenovirus. Amplified cDNAs are cloned, sequenced and aligned, amongst others, against existing Genbank sequences.

2) cDNA Expression Library Screening

cDNA libraries, either commercially available or generated using a CAR-negative cell line which is highly transducible with chimaeric adenoviruses carrying the fiber protein of members of for example subgroup D or subgroup F, are used for expression library screening using either radiolabelled adenovirus or recombinant produced knob proteins as probes. Clones or plaques which bind to the probe are picked, amplified and re-tested for enrichment of probe binding. Finally phages are picked after which the cDNA content is elucidated by sequence analysis. Retrieved cDNAs are cloned, sequenced and aligned, amongst others, against existing Genbank sequences.

3) Peptidase Treatment of Cells After Adenovirus Binding

Cells which are highly transducible with chimaeric adenoviruses carrying the fiber protein of members of for example subgroup D, are treated with different peptidases after binding of the chimaeric adenovirus. The panel of peptidases suited is first tested on the chimaeric adenovirus only to ensure that capsid proteins of the chimaeric virus is not cleaved. Peptidae treated cells are spun down after which the supernatant is added to 24-well plates precoated with anti-adenovirus hexon and/or penton antibodies. After binding of adenovirus to the precoated plastic support, wells are washed extensively with PBS. Upon washing, the adenovirus is harvested after which either protein gel electrophoresis or Malditoff is used to identify whether parts of a cellular protein is bound to the fiber protein or whether extra protein bands are visible as compared to protein gel electrophoresis or Malditoff of a purified batch of adenovirus only. As a negative control for the above described experiments cells negative for infection with a chimaeric adenovirus carrying a fiber of a member of subgroup D can be used. Alternatively, cells which are highly transducible with chimaeric adenoviruses carrying the fiber protein of members of for example subgroup D, are first treated with peptidases after which the medium is incubated with adenoviruses bound to a plastic support.

The above described examples encompasses the construction of recombinant adenoviral vectors chimaeric for the fiber protein which results in an altered infection hostrange. The alteration of the infection host range results in highly efficient infection of cells negative for the CAR protein which is the protein required by adenovirus serotype 5 for efficient infection. These vectors are generated for the purpose of gene transfer and recombinant DNA vaccines. These vectors are thus ideally suited for gene transfer to tissues, and/or organs of which de cells do not express detectable levels of CAR.

Figure and Table Legends

Table 1: Association of human adenovirus serotypes with human disease.

Table 2: Oligonucleotides and degenerate oligonucleotides used for the amplification of DNA encoding for fiber protein derived from alternative human adenovirus serotypes. Bold letters in oligonucleotides A-E represent an NdeI restriction site. Bold letters in oligonucleotides 1-6 and 8 represent an NsiI restriction site. Bold letters in oligonucleotide 7 represents a PacI restriction site.

Table 3: Production results of fiber chimaeric adenoviruses. The number of virus particles per ml were determined using HPLC. The number of infectious units (IU) per millilitre were determined through titration on human 911 cells. For infection experiments, the number of virus particles per millilitre is taken from all chimaeric adenoviruses since IU/ml reflects a receptor mediated process.

Table 4: Flow cytometric results on expression of integrins αᵥβ3 and αᵥβ5, the Coxsacki adenovirus receptor (CAR), and MHC class I on the membranes of human cell lines and human primary cells. A549: Human lung carcinoma cell line (ATCC, CCL-1185). K562: Human erythroid leukemia (ATCC, CCL-243). SupT1: Human Lymphoblast hybrid B and T (ATCC, CRL-1991). GM09503: Human primary fibroblasts. HEPG2: Human liver carcinoma (ATCC, HB8065). CEM: human lymphoblast cells (ATCC, CRL-1992). HeLa: Human cervix carcinoma (ATCC, CCL-2). Primary amniocytes and chorion villi cells were obtained from department of antropogenetics, Leiden, The Netherlands. Primary Smooth muscle cells, Human umbilical vein endothelial cells, and synoviocytes were obtained from TNO-PG, Leiden, The Netherlands. Shown is the percentage of cells expressing either molecule on their membrane. ND: not determined. 0% means undetectable expression of the molecule on the membrane of the cell using flow cytometry. 100% means high expression of the molecule on the cell membrane.

FIG. 1: Schematic presentation of adapter plasmid pMLPI.TK.

FIG. 2: Schematic presentation of adapter plasmid pAd/L420-HAS.

FIG. 3: Schematic presentation of adapter plasmid pAd5/CLIP

FIG. 4: Schematic presentation of plasmid system which requires only one recombinational event to generate recombinant adenoviruses.

FIG. 5: Schematic presentation of plasmid system which requires two recombinational events to generate recombinant adenoviruses.

FIG. 6: Schematic presentation of generation of plasmid pBr/AdBamRDeltaFib in which the Adenovirus type 5 fiber DNA is replaced by a short DNA stretch containing an unique NsiI site.

FIG. 7: Fiber protein sequences of adenovirus serotypes 8, 9, 13, 14, 20, 23, 24, 25, 27, 28, 29, 30, 32, 33, 34, 35, 36, 37, 38, 39, 42, 43, 44, 45, 46, 47, 48, 49, and 51. Bold letters represent part of the tail of adenovirus serotype 5. If bold letters not present it means that a PCR fragment was sequenced which does not contain the Ad5 tail. An X, present in the sequence means unidentified amino acid due to unidentified nucleotide. At the end of the sequence the stop codon of the fiber is presented by a dot.

FIG. 8: Transduction of human lung carcinoma cells (A549) with a panel of chimaeric adenoviruses carrying the fiber of adenovirus 12, 16, 17, 28, 32, 40-L, or 51. Adenovirus 5 served as reference. Cells were infected with increasing MOI based on virus particles per cell: 10, 50, 250, 1250, 2500 (see legend on the right of graph). Luciferase transgene expression is expressed as relative light units (RLU) per μg of protein.

FIG. 9: Transduction of human erythroid leukemia cells (K562) with a panel of chimaeric adenoviruses carrying the fiber of adenovirus 12, 16, 28, 32, 40-S, 40-L, 49, or 51. Adenovirus 5 served as reference. Cells were infected with increasing MOI based on virus particles per cell: 10, 50, 250, 1250, 2500, 5000 (see legend on the right of graph). Luciferase transgene expression is expressed as relative light units (RLU) per μg of protein. Error bars represent SD.

FIG. 10: Transduction of human primary fibroblasts (GM09503) with a panel of chimaeric adenoviruses carrying the fiber of adenovirus 12, 16, 28, 32, 40-L, 49, or 51. Adenovirus 5 served as reference. Cells were infected with increasing MOI based on virus particles per cell: 10, 50, 250, 1250, 2500, 5000 (see legend on the right of graph). Luciferase transgene expression is expressed as relative light units (RLU) per μg of protein. Error bars represent SD.

FIG. 11: Transduction of human lymphoblast cells (SupT1) with a panel of chimaeric adenoviruses carrying the fiber of adenovirus 12, 16, 28, 32, 40-S, 40-L, 49, or 51. Adenovirus 5 served as reference. Cells were infected with increasing MOI based on virus particles per cell: 10, 50, 250, 1250, 2500, 5000 (see legend on the right of graph). Luciferase transgene expression is expressed as relative light units (RLU) per μg of protein. Error bars represent SD.

FIG. 12: Transduction of human chorion villi cells with a panel of chimaeric adenoviruses carrying the fiber of adenovirus 12, 16, 28, 32, 40-L, 49, or 51. Adenovirus 5 served as reference. Cells were infected with increasing MOI based on virus particles per cell: 10, 50, 250, 1250, 2500, 5000 (see legend on the right of graph). Luciferase transgene expression is expressed as relative light units (RLU) per μg of protein. Error bars represent SD.

FIG. 13: Transduction of human hepatic cells (HEPG2) with a panel of chimaeric adenoviruses carrying the fiber of adenovirus 12, 16, 28, 32, 40-S, 40-L, 49, or 51. Adenovirus 5 served as reference. Cells were infected with increasing MOI based on virus particles per cell: 10, 50, 250, 1250, 2500, 5000 (see legend on the right of graph). Luciferase transgene expression is expressed as relative light units (RLU) per μg of protein. Error bars represent SD.

### Reference

Arnberg N., Mei Y. and Wadell G., 1997. Fiber genes of adenoviruses with tropism for the eye and the genital tract. Virology 227: 239-244.

Bout A., 1997. Gene therapy, p. 167-182. In: D. J. A. Crommelin and R. D. Sindelar (ed.), Pharmaceutical biotechnology Harwood Academic Publishers.

Bout, A. 1996. Prospects for human Gene therapy. Eur. J. drug Met. And Pharma. 2, 175-179.

Blaese et al., Cancer Gene Ther., 2 (1995):291-297).

Brody and Crystal, Ann. N. Y. Acad. Sci. 716(1994):90-101.

Chroboczek J., Ruigrok R. W. H., and Cusack S., 1995. Adenovirus fiber, p. 163-200. In: W. Doerfler and P. Bohm (ed.), The molecular repertoire of adenoviruses, I. Springer-Verlag, Berlin.

Defer C., Belin M., Caillet-Boudin M. and Boulanger P., 1990. Human adenovirus-host cell interactions; comparative study with members of subgroup B and C. Journal of Virology 64 (8): 3661-3673.

De Jong, J. C., Wermenbol, A. G., Verweij-Uijterwaal, M. W., Slaterus, K. W., Wertheim-van Dillen, P., van Doornum, G. J. J., Khoo, S. H., and Hierholzer, J. C. (1998) Adenoviruses from HIV-infected patients, including two new candidate serotypes Ad50 and Ad51 of Subgenus D and B1 respectively. In preparation.

Eisenlohr, L. C., Gerard, W., and Hackett, C. J. (1987). Role of receptor-binding activity of the viral hemagglutin molecule in the presentation of influenza virus antigens to helper T-cells. J. Virol 61, 1375-1383

Eiz B and Pring-okerblom P., 1997. Molecular characterization of the type-specific g-determinant located on the adenovirus fiber. Journal of Virology 71: 6576-6581.

Francki, R. I. B., Fauquet, C. M., Knudson, D. L. and Brown, F. (1991) Classification and nomenclature of viruses. Fifth report of the international Committee on taxonomy of viruses. Arch. Virol. Suppl. 2, 140-144

GahÈry-SÈgard H., Farace F., Godfrin D., Gaston J., Lengagne R., Tursz T., Boulanger P. and Guillet J., 1998.Immune response to recombinant capsid proteins of adenovirus in humans: antifiber and anti-penton base antibodies have a synergistic effect on neutralizing activity. Journal of Virology 72: 2388-2397.

Gall J., Kass-Eisler A., Leinwand L. and Falck-Pedersen E., 1996. Adenovirus type 5 and 7 capsid chimera: fiber replacement alters receptor tropism without affecting primary immune neutralisation epitopes. Journal of Virology 70 (4): 2116-2123.

Greber, U. F., Willets, M., Webster, P., and Helenius, A. (1993). Stepwise dismanteling of adenovirus 2 during entry into cells. Cell 75, 477-486.

Hynes, R. O. (1992) Integrins: versatility, modulation and signalling in cell adhesion. Cell 69, 11-25

Herz and Gerard, Proc. Natl. Acad. Sci. U.S.A., 96 (1993):2812-2816

Hierholzer, J. C. (1992) Adenovirus in the immunocompromised host. Clin. Microbiol Rev. 5, 262-274.

Hierholzer, J. C., Wigand, R., Anderson, L. J., Adrian, T., and Gold, J. W. M. (1988) Adenoviruses from patients with AIDS: a plethora of serotypes and a description of five new serotypes of subgenus D (types43-47). J. Infect. Dis. 158, 804-813.

Ishibashi, M. and Yasue (1983) in Adenoviruses of Animals, Chapter 12, p497-561

Kay, R., Takei, F., and Humphries, R. K. (1990). Expression cloning of a cDNA encoding M1/69-J11d heat-stable antigens. J. Immunol. 145 (6), 1952-1959

Khoo, S. H., Bailey, A. S., De Jong, J. C., and Mandal, B. K. (1995). Adenovirus infections in human immunodeficiency virus-positive patients: Clinical features and molecular epidemiology. J. Infect. Dis 172, 629-637

Kidd, A. H., Chrboczek, J., Cusack, S., and Ruigrok, R. W. |H. (1993) Adenovirus type 40 virions containtwo distinct fibers. Virology 192, 73-84.

Krasnykh V. N., Mikheeva G. V., Douglas J. T. and Curiel D. T., 1996.Generation of recombinant adenovirus vectors with modified fibers for altering viral tropism. Journal of Virology 70(10): 6839-6846.

Krasnykh V., Dmitriev I., Mikheeva G., Miller C. R., Belousova N. and Curiel D. T.,1998.

Characterization of an adenovirus vector containing a heterologous peptide epitope in the HI loop of the fiber knob. Journal of Virology 72(3): 1844-1852.

Leopold, P. L., Ferris, B., Grinberg, I., Worgall, S., Hackett, N. R., and Crystal, R. G. (1998). Fluorescent virions: Dynamic tracking of the pathway of adenoviral vectors in living cells. Hum. Gene Ther. 9, 367-378.

Levrero, M., Barban, V., Manteca, S., Ballay, A., Balsamo, C., Avantaggiata, M. L., Natoli, G., Skellekens, H., Tiollais, P., and Perricaudet, M. (1991(. Defective and non-defective adenovirus vectors for expression foreign genes in vitro and in vivo. Gene 101, 195-202.

Matlin, K. S., Reggio, H., Helenius, A., and Simons, K. (1981). Infectious entry pathway of influenza virus in a canine kidney cell line. J. Cell Biol. 91, 601-613

Morgan, C., Rozenkrantz, H. S., and Mednis, B. (1969(Structure and development of viruses as observed in the electron microscope.X. Entry and uncoating of adenovirus. J.Virol 4, 777-796.

Roelvink, P. W., Lizonova, A., Lee, J. G. M., Li, Y., Bergelson, J. M. Finberg, R. W., Brough, D. E., Kovesdi, I., and Wickham, T. J. (1998). The Coacksackievirus-adenovirus receptor protein can function as a cellular attachment protein for adenovirus serotypes from subgroups A,C,D,E, and F. J. Virology 72 (No. 10), 7909-7915

Richman, D. D., Hostetler, K. Y., Yazaki, P. J., and Clark, S. (1986). Fate of influenza A virion proteins after entry into subcellular fractions of LLC cells and the effect of amantadine. Virology 151, 200-210

Stevenson S. C., Rollence M., White B., Weaver L. and McClelland A., 1995.

Human adenovirus serotypes 3 and 5 bind to two different cellular receptors via the fiber head domain. Journal of Virology 69(5): 2850-2857.

Stevenson S. C., Rollence M., Marshall-Neff J. and McClelland A., 1997.Selective targeting of human cells by a chimaeric adenovirus vector containing a modified fiber protein. Journal of Virology 71(6): 4782-4790.

Signas, G., Akusjarvi, G., and Petterson, U. (1985). Adenovirus 3 fiberpolypeptide gene: Complications for the structure of the fiber protein. J. Virol. 53, 672-678.

Stouten, P. W. F., Sander, C., Ruigrok, R. W. H., and Cusack, S. (1992) New triple helical model for the shaft of the adenovirus fiber. J. Mol. Biol. 226, 1073-1084.

Schulick, A. H., Vassalli, G., Dunn, P. F., Dong, G., Rade, J. J., Zamarron, C. and Dichek, D. A. (1997). Established immunity precludes adenovirus-mediated gene transfer inrat carotid arteries.

Schnurr, D and Dondero, M. E. (1993) Two new candidate adenovirus serotypes Intervirol. 36, 79-83

Svensson, V. and Persson, R. (1984). Entry of adenovirus 2 into Hela cells. J. Virol. 51, 687-694.

Varga, M. J., Weibull, C., and Everitt, E. (1991). Infectious entry pathway of adenovirus type 2. J. Virol 65, 6061-6070.

Wickham T. J., Carrion M. E. and Kovesdi I., 1995. Targeting of adenovirus penton base to new receptors through replacement of its RGD motif with other receptor-specific peptide motifs. Gene Therapy 2: 750-756.

Wickham T. J., Segal, D. M., Roelvink, P. W., Carrion M. E., Lizonova, A., Lee, G-M., and Kovesdi, I. (1996). Targeted adenovirus gene transfer to endothelial and smooth muscle cells by using bispecific antibodies. J. Virol. 70 (10), 6831-6838

Wickham, T. J., Mathias, P., Cherish, D. A., and Nemerow, G. R. (1993) Integrins avb3 and avb5 promote adenovirus internalisation but not virus attachment. Cell 73, 309-319.

Tables and Figures

TABLE 1

| Syndrom | Subgenus | Serotype |
|---|---|---|
| Respiratory illness | A | 31 |
| | B | 3, 7, 11, 14, 21, 34, 35, 51 |
| | C | 1, 2, 5, 6 |
| | D | 39, 42-48 |
| | E | 4 |
| Keratoconjunctivitis | B | 11 |
| (eye) | D | 8, 19, 37, 50 |
| Hemorrhagic cystitis | B | 7, 11, 14, 16, 21, 34, 35 |
| (Kidney) | | |
| And urogenital tract | C | 5 |
| infections | D | 39, 42-48 |
| Sexual transmission | C | 2 |
| | D | 19, 37 |
| Gastroenteritis | A | 31 |
| | B | 3 |
| | C | 1, 2, 5 |
| | D | 28 |
| | F | 40, 41 |
| CNS disease | A | 12, 31 |
| | B | 3, 7 |
| | C | 2, 5, 6 |
| | D | 32, 49 |
| Hepatitis | A | 31 |
| | C | 1, 2, 5 |
| Disseminated | A | 31 |
| | B | 3, 7, 11, 21 |
| | D | 30, 43-47 |
| None (???) | A | 18 |
| | D | 9, 10, 13, 15, 17, 20, 22-29, 33, 36, 38 |

TABLE 2

| Serotype | Tail oligonucleotide | Knob oligonucleotide |
|---|---|---|
| 4 | A | 1 |
| 8 | B | 2 |
| 9 | B | 2 |
| 12 | E | 3 |
| 16 | C | 4 |
| 19p | B | 2 |
| 28 | B | 2 |
| 32 | B | 2 |
| 36 | B | 2 |
| 37 | B | 2 |
| 40-1 | D | 5 |
| 40-2 | D | 6 |
| 41-s | D | 5 |
| 41-1 | D | 7 |
| 49 | B | 2 |
| 50 | B | 2 |
| 51 | C | 8 |

| | |
|---|---|
| A: | 5′-CCC CTG TAT CCA TAT GAT GCA GAC AAC GAC CGA |

CC-3′

| | |
|---|---|
| B: | 5′-CCC GTC TAC CCA TAT GGC TAC GCG CGG-3′ |
| C: | 5′-CCK GTS TAC CCA TAT GAA GAT GAA AGC-3′ |
| D: | 5′-CCC GTC TAC CCA TAT GAC ACC TYC TCA ACT C-3′ |
| E: | 5′-CCC GTT TAC CCA TAT GAC CCA TTT GAC ACA TCA |

GAC-3′

| | |
|---|---|
| 1: | 5″-CCG ATG CAT TTA TTG TTG GGC TAT ATA GGA-3′ |
| 2: | 5′-CCG ATG CAT TYA TTC TTG GGC RAT ATA GGA-3′ |
| 3: | 5′-CCG ATG CAT TTA TTC TTG GGR AAT GTA WGA AAA |

GGA-3′

| | |
|---|---|
| 4: | 5′-CCG ATG CAT TCA GTC ATC TTC TCT GAT ATA-3′ |
| 5: | 5′-CCG ATG CAT TTA TTG TTC AGT TAT GTA GCA-3′ |
| 6: | 5′-GCC ATG CAT TTA TTG TTC TGT TAC ATA AGA-3′ |
| 7: | 5′-CCG TTA ATT AAG CCC TTA TTG TTC TGT TAC ATA |

AGA A-3′

| | |
|---|---|
| 8: | 5′-CCG ATG CAT TCA GTC ATC YTC TWT AAT ATA-3′ |

TABLE 3

| Adenovirus | Virus particles/ml | Infectious units/ml |
|---|---|---|
| Ad5Fib5 | 2.2 × 10¹² | 6.8 × 10¹¹ |
| Ad5Fib12 | 4.4 × 10¹² | 1.9 × 10¹² |
| Ad5Fib16 | 1.4 × 10¹² | 3.0 × 10¹⁰ |
| Ad5Fib17 | 9.3 × 10¹¹ | 9.5 × 10⁹ |
| Ad5Fib28 | 5.4 × 10¹⁰ | 2.8 × 10⁸ |
| Ad5Fib32 | 2.0 × 10¹² | 1.1 × 10¹² |
| Ad5Fib40-S | 3.2 × 10¹⁰ | 1.0 × 10¹⁰ |
| Ad5Fib40-L | 2.0 × 10¹² | 6.4 × 10¹¹ |
| Ad5Fib49 | 1.2 × 10¹² | 4.3 × 10¹¹ |
| Ad5Fib51 | 5.1 × 10¹² | 1.0 × 10¹² |

TABLE 4

| Cell line | αᵥβ3 | αᵥβ5 | CAR | MHC class I |
|---|---|---|---|---|
| A549 | 17% | 98% | 100% | ND |
| K562 | 12% | 55% | 0% | 15% |
| GM09503 | 20% | 50% | 0% | 100% |
| CEM | 0% | 0% | 3% | 100% |
| SupT1 | 5% | 1% | 70% | 100% |
| Smooth muscle | 100% | 70% | 0% | 15% |
| cells | | | | |
| HUVEC | 100% | 15% | 10% | 90% |
| Synoviocytes | 30% | 40% | 0% | 100% |
| 1⁰ chorionvilli | 100% | 0% | 12% | 100% |
| HepG2 | 0% | 10% | 100% | 80% |

What is claimed is:

1. A method of delivering a nucleic acid of interest to a host cell by means of a gene delivery vehicle based on adenoviral material, said method comprising:
delivering, with said gene delivery vehicle, the nucleic acid of interest to the host cell by associating the gene delivery vehicle with a binding site and/or a receptor present on at least one CAR-negative cell, said binding site and/or receptor being a binding site and/or a receptor for adenovirus subgroup D and/or adenovirus subgroup F.

2. A method of delivering a nucleic acid of interest to a CAR-negative cell, said method comprising:
contacting said CAR-negative cell with a gene delivery vehicle comprising:
the nucleic acid of interest and
adenoviral material involved in binding to a host cell, said adenoviral material being from adenovirus subgroup D and/or adenovirus subgroup F.

3. A chimeric gene delivery vehicle based on at least two adenoviruses, wherein a cell recognizing element of said chimeric gene delivery vehicle is based on adenoviral material from adenovirus subgroup D and/or adenovirus subgroup F, which adenoviral material confers the capability of infecting CAR-negative cells.

4. The chimeric gene delivery vehicle of claim 3, wherein said adenoviral material is based on a fiber, penton and/or hexon protein of adenovirus subgroup D and/or adenovirus subgroup F.

5. The chimeric gene delivery vehicle of claim 3 or claim 4, further comprising:
an element from adenovirus 35 responsible for at least partially avoiding an immune response against adenovirus 35 in man.

6. The chimeric gene delivery vehicle of any one of claims 3-5, comprising an adenoviral 16 element or a functional analogue thereof, said adenoviral 16 element conferring adenovirus 16 with an enhanced capability to infect smooth muscle cells and/or synoviocytes.

7. The chimeric gene delivery vehicle of any one of claims 3-6, further comprising adenoviral nucleic acid.

8. The chimeric gene delivery vehicle of any one of claims 3-7, further comprising adenoviral nucleic acid derived from at least two different adenoviral types.

9. The chimeric gene delivery vehicle of claim 7 or claim 8, wherein said adenoviral nucleic acid comprises at least one sequence encoding a capsid protein comprising at least a tissue tropism determining fragment of adenovirus subgroup D and/or adenovirus subgroup F capsid protein.

10. The chimeric gene delivery vehicle of claim 7, claim 8, or claim 9, wherein said adenoviral nucleic acid is modified to reduce or disable the ability of said adenoviral nucleic acid to replicate in a target cell.

11. The chimeric gene delivery vehicle of claim 7, claim 8, claim 9, or claim 10, wherein said adenoviral nucleic acid has been modified to reduce or disable the capacity of a host immune system to mount an immune response against adenoviral proteins encoded by said adenoviral nucleic acid.

12. The chimeric gene delivery vehicle of claim 7, claim 8, claim 9, claim 10, or claim 11, comprising a minimal adenovirus vector or an integrating adenovirus.

13. The chimeric gene delivery vehicle of any one of the claims 1-12, further comprising at least one non-adenoviral nucleic acid.

14. The chimeric gene delivery vehicle of any one of claims 7-13, wherein said adenoviral nucleic acid is produced by a process comprising:
welding together, through homologous recombination, two nucleic acid molecules comprising partially overlapping sequences wherein said partially overlapping sequences allowing essentially only a single homologous recombination event thus generating a physically linked nucleic acid comprising:
a nucleic acid of interest, at least two functional adenoviral inverted terminal repeats (ITRs), and a functional encapsulation signal, or functional parts, derivatives or analogues of said ITRs and/or encapsulation signal.

15. A cell for producing the chimeric gene delivery vehicle of any one of the claims 3-14, said cell comprising:
first means for assemblying said gene delivery vehicle wherein said first means includes further means for producing of an adenovirus capsid protein, said capsid protein comprising at least a receptor and/or binding site binding fragment of adenovirus subgroup D and/or adenovirus subgroup F capsid protein.

16. The cell of claim 15, wherein said cell is of a PER.C6 cell (ECACC deposit number 96022940) origin.

17. A pharmaceutical composition comprising the chimeric gene delivery vehicle of any one of claims 1 through 14.

18. A receptor and/or a binding site for adenovirus subgroup D and/or adenovirus subgroup F, associated with CAR-negative cells.

19. The receptor and/or a binding site of claim 18, present on K562 cells, amniotic fluid cells and/or primary fibroblast cells.

20. A capsid protein derived from a adenovirus subgroup D and/or adenovirus subgroup F or a functional part, derivative and/or analogue thereof.

21. The capsid protein of claim 20, wherein said capsid protein is a fiber protein.

22. An isolated and/or recombinant nucleic acid encoding a capsid protein of claim 20 or claim 21.

23. The isolated and/or recombinant nucleic acid of claim 22, wherein said isolated and/or recombinant nucleic acid comprises a sequence selected from the group of sequences depicted in FIG. 7.
